(19) United States
(12) Patent Application Publication
(10) Pub. No.: US 2026/0345864 A1
(43) Pub. Date: Nov. 5, 2026

(54) COMPOSITIONS AND METHODS FOR IMPROVING OVARIAN FUNCTION

(71) Applicant: Gameto, Inc., New York, NY (US)

(72) Inventors: Dina RADENKOVIC, New York, NY (US); Christian KRAMME, New York, NY (US); Martin VARSAVSKY, New York, NY (US)

(21) Appl. No.: 19/102,710

(22) PCT Filed: Aug. 14, 2023
(86) PCT No.: PCT/US2023/030167

§ 371 Date: Feb. 10, 2025

Related U.S. Application Data
(63) Continuation of application No. 17/886,773, filed on Aug. 12, 2022, now Pat. No. 11,744,920.

(51) Int. Cl. A61L 27/38; A61K 9/00; A61K 35/12; A61L 27/24; A61L 27/36; C12N 5/00

(52) U.S. Cl. CPC A61L 27/38; A61K 9/0024; A61K 35/12; A61L 27/24; A61L 27/3633; C12N 5/0012; C12N 5/0068; C12N 2533/90

(57) ABSTRACT

Featured are compositions and methods for an ovarian implant comprising ovarian support cells. In particular, the disclosure features methods of culturing ovarian support cells for the purpose of implantation into a subject in need thereof. Additional methods for administering the OSC implant to the subject are provided. Such methods and compositions are particularly useful for assisted reproduction technology (ART) procedures and hormone replacement therapy (HRT).

### TECHNICAL FIELD

This disclosure relates to the field of hormone replacement therapy, particularly in the context of an implant for treating, ameliorating, or altogether reversing ovarian decline.

### BACKGROUND

One in ten women struggle with infertility, requiring assisted reproductive technology (ART) such as in vitro fertilization (IVF). Challenges remain with maintaining oocyte health in culture, resulting in low oocyte quality and subsequently poor embryo quality. Furthermore, oocytes that are developmentally immature are traditionally discarded, constricting the available oocyte pool for IVF. In vitro maturation (IVM) holds the promise to mature oocytes in vitro after egg extraction, allowing for utilization of all retrieved eggs. Current methods for IVM are inefficient, using follicle-stimulating hormone (FSH) spike-in to the culture media, showing 5-40% maturation of immature eggs. Even worse, this method results in many unhealthy eggs, with an embryo viability rate under 17%, far lower than for standard IVF. Thus, there remains a need in the field for promoting oocyte maturation for a female subject undergoing an ART procedure.

### SUMMARY OF THE INVENTION

In a first aspect, the disclosure features an implant containing (i) a population of ovarian support cells (OSCs) and (ii) an extracellular matrix substrate.

In some embodiments, the population of OSCs includes one or more ovarian granulosa cells, ovarian lutein cells, and/or ovarian theca cells.

In some embodiments, the population of OSCs contains one or more ovarian granulosa cells. In some embodiments, the one or more ovarian granulosa cells express one or more, or all, of proteins FOXL2, CD82, follicle-stimulating hormone receptor (FSHR), FOXL2, NR5A1, GATA4, RUNX1, and RUNX2.

In some embodiments, the implant contains from about 1×10⁶ and to about 1×10⁷ ovarian granulosa cells. In some embodiments, the implant contains from about 1×10⁶ to about 2×10⁶ ovarian granulosa cells, from about 2×10⁶ to about 3×10⁶ ovarian granulosa cells, from about 3×10⁶ to about 4×10⁶ ovarian granulosa cells, from about 4×10⁶ to about 5×10⁶ ovarian granulosa cells, from about 5×10⁶ to about 6×10⁶ ovarian granulosa cells, from about 6×10⁶ to about 7×10⁶ ovarian granulosa cells, from about 7×10⁶ to about 8×10⁶ ovarian granulosa cells, from about 8×10⁶ to about 9×10⁶ ovarian granulosa cells, or from about 9×10⁶ to about 1×10⁷ ovarian granulosa cells. In some embodiments, the implant contains about 1×10⁶ ovarian granulosa cells, about 2×10⁶ ovarian granulosa cells, about 3×10⁶ ovarian granulosa cells, about 4×10⁶ ovarian granulosa cells, about 5×10⁶ ovarian granulosa cells, about 6×10⁶ ovarian granulosa cells, about 7×10⁶ ovarian granulosa cells, about 8×10⁶ ovarian granulosa cells, about 9×10⁶ ovarian granulosa cells, or about 1×10⁷ ovarian granulosa cells.

In some embodiments, the one or more ovarian granulosa cells secrete an estrogen. In some embodiments, the estrogen is estradiol.

In some embodiments, the population of OSCs includes one or more ovarian lutein cells. In some embodiments, the one or more ovarian lutein cells express one or more, or all, of proteins KRT19, CYP19A1, STAR, CYP17A1, and PGR.

In some embodiments, the implant contains from about 1×10⁶ and to about 1×10⁷ ovarian lutein cells. In some embodiments, the implant contains from about 1×10⁶ to about 2×10⁶ ovarian lutein cells, from about 2×10⁶ to about 3×10⁶ ovarian lutein cells, from about 3×10⁶ to about 4×10⁶ ovarian lutein cells, from about 4×10⁶ to about 5×10⁶ ovarian lutein cells, from about 5×10⁶ to about 6×10⁶ ovarian lutein cells, from about 6×10⁶ to about 7×10⁶ ovarian lutein cells, from about 7×10⁶ to about 8×10⁶ ovarian lutein cells, from about 8×10⁶ to about 9×10⁶ ovarian lutein cells, or from about 9×10⁶ to about 1×10⁷ ovarian lutein cells. In some embodiments, the implant contains about 1×10⁶ ovarian lutein cells, about 2×10⁶ ovarian lutein cells, about 3×10⁶ ovarian lutein cells, about 4×10⁶ ovarian lutein cells, about 5×10⁶ ovarian lutein cells, about 6×10⁶ ovarian lutein cells, about 7×10⁶ ovarian lutein cells, about 8×10⁶ ovarian lutein cells, about 9×10⁶ ovarian lutein cells, or about 1×10⁷ ovarian lutein cells.

In some embodiments, the one or more ovarian lutein cells secrete a progestogen. In some embodiments, the progestogen is progesterone.

In some embodiments, the population of OSCs includes one or more ovarian theca cells. In some embodiments, the one or more ovarian theca cells express one or both of proteins NR2F2 and GATA4.

In some embodiments, the implant contains from about 1×10⁶ and to about 1×10⁷ ovarian theca cells. In some embodiments, the implant contains from about 1×10⁶ to about 2×10⁶ ovarian theca cells, from about 2×10⁶ to about 3×10⁶ ovarian theca cells, from about 3×10⁶ to about 4×10⁶ ovarian theca cells, from about 4×10⁶ to about 5×10⁶ ovarian theca cells, from about 5×10⁶ to about 6×10⁶ ovarian theca cells, from about 6×10⁶ to about 7×10⁶ ovarian theca cells, from about 7×10⁶ to about 8×10⁶ ovarian theca cells, from about 8×106 to about 9×106 ovarian theca cells, or from about 9×106 to about 1×10⁷ ovarian theca cells. In some embodiments, the implant contains about 1×10⁶ ovarian theca cells, about 2×10⁶ ovarian theca cells, about 3×10⁶ ovarian theca cells, about 4×10⁶ ovarian theca cells, about 5×10⁶ ovarian theca cells, about 6×10⁶ ovarian theca cells, about 7×10⁶ ovarian theca cells, about 8×10⁶ ovarian theca cells, about 9×10⁶ ovarian theca cells, or about 1×10⁷ ovarian theca cells.

In some embodiments, the one or more ovarian theca cells secrete an androgen. In some embodiments, the androgen is androstenedione.

In some embodiments, the population of OSCs includes one or more ovarian granulosa cells, ovarian lutein cells, and ovarian theca cells, and the ratio of ovarian granulosa cells to ovarian lutein cells to ovarian theca cells in the implant is from about 10:1:1 to about 1,000:1:1. In some embodiments, the ratio of ovarian granulosa cells to ovarian lutein cells to ovarian theca cells in the implant is about 100:1:1. In some embodiments, the one or more OSCs are obtained by differentiating a population of induced pluripotent stem cells (iPSCs) into one or more ovarian granulosa cells, ovarian lutein cells, and/or ovarian theca cells.

In some embodiments, the extracellular matrix substrate comprises one or more of collagen, an epidermal growth factor (EGF), an elastin, a fibronectin, a vitronectin, a laminin, a cell adhesion protein, or a plant-derived protein or protein polymer. In some embodiments, the collagen is fibrillar collagen. In some embodiments, the collagen is collagen type I, type II, type III, type V, type XI. In some embodiments, the elastin is tropoelastin or mature elastin. In some embodiments, the plant-derived protein polymer is alginate.

In another aspect, the disclosure features a method of increasing secretion of an estrogen, progestogen, and/or androgen in a human subject, the method including administering to the subject the implant of any of the above aspects or embodiments of the disclosure.

In another aspect, the disclosure features a method of restoring secretion of an estrogen (e.g., estradiol), progestogen (e.g., progesterone), and/or androgen (e.g., androstenedione) in a post-menopausal human subject, the method including administering to the subject the implant of any of the above aspects or embodiments of the disclosure.

In another aspect, the disclosure features a method of treating a human subject diagnosed as having ovarian dysfunction, the method including administering to the subject the implant of any of the above aspects or embodiments of the disclosure.

In some embodiments, prior to administration of the implant to the subject, the subject exhibits consistent, circulating estradiol levels of about 20 pg/ml or less, optionally wherein the subject exhibits consistent, circulating estradiol levels of from about 10 to about 20 pg/ml. In some embodiments, prior to administration of the implant to the subject, the subject exhibits consistent, circulating progesterone levels of about 0.5 ng/ml or less, optionally wherein the subject exhibits consistent, circulating progesterone levels of from about 0.01 ng/ml to about 0.5 ng/ml.

In some embodiments, prior to administration of the implant to the subject, the subject has previously undergone an ovariectomy.

In some embodiments, prior to administration of the implant to the subject, the subject exhibits ovarian decline. In some embodiments, the ovarian decline is a result of menopause. In some embodiments, the ovarian decline is not a result of menopause, optionally wherein the menopause is premature menopause. In some embodiments, the ovarian decline is a result of a disease or condition selected from primary ovarian insufficiency (POI), polycystic ovarian syndrome (PCOS), endometriosis, uterine fibroids, gynecological cancer, interstitial cystitis, pelvic inflammatory disease (PID), vaginitis, cervical dysplasia, and pelvic floor prolapse.

In some embodiments, upon administration of the implant to the subject, the population of OSCs secretes estradiol during an early- to mid-follicular phase of the subject's menstrual cycle at a serum concentration of from about 20 pg/ml to about 80 pg/ml. In some embodiments, upon administration of the implant to the subject, the population of OSCs secretes estradiol during an early- to mid-follicular phase of the subject's menstrual cycle at a serum concentration of from about 20 pg/ml to about to 30 pg/ml, from about 30 pg/ml to about 40 pg/ml, from about 40 pg/ml to about 50 pg/ml, from about 50 pg/ml to about 60 pg/ml, from about 60 pg/ml to about 70 pg/ml, or from about 70 pg/ml to about 80 pg/ml.

In some embodiments, upon administration of the implant to the subject, the population of OSCs secretes estradiol during a mid- to late-follicular phase of the subject's menstrual cycle at a serum concentration of from about 20 pg/ml to about 200 pg/ml. In some embodiments, upon administration of the implant to the subject, the population of OSCs secretes estradiol during a mid- to late-follicular phase of the subject's menstrual cycle at a serum concentration of from about 20 pg/ml to about to 50 pg/ml, from about 50 pg/ml to about 70 pg/ml, from about 70 pg/ml to about 90 pg/ml, from about 90 pg/ml to about 110 pg/ml, from about 110 pg/ml to about 130 pg/ml, from about 130 pg/ml to about 150 pg/ml, from about 150 pg/ml to about t 170 pg/ml, or from about 170 pg/ml to about 200 pg/ml.

In some embodiments, upon administration of the implant to the subject, the population of OSCs secretes estradiol during a pre-ovulation phase of the subject's menstrual cycle at a serum concentration of from about 100 pg/ml to about 400 pg/ml. In some embodiments, administration of the implant to the subject, the population of OSCs secretes estradiol during a pre-ovulation phase of the subject's menstrual cycle at a serum concentration of from about 100 pg/ml to about to 150 pg/ml, from about 150 pg/ml to about 200 pg/ml, from about 200 pg/ml to about 250 pg/ml, from about 250 pg/ml to about 300 pg/ml, from about 300 pg/ml to about 350 pg/ml, or from about 350 pg/ml to about 400 pg/ml.

In some embodiments, upon administration of the implant to the subject, the population of OSCs secretes estradiol during an ovulation phase of the subject's menstrual cycle at a serum concentration of from about 250 pg/ml to about 500 pg/ml. In some embodiments, upon administration of the implant to the subject, the population of OSCs secretes estradiol during an ovulation phase of the subject's menstrual cycle at a serum concentration of from about 250 pg/ml to about to 300 pg/ml, from about 300 pg/ml to about 350 pg/ml, from about 350 pg/ml to about 400 pg/ml, from about 400 pg/ml to about 450 pg/ml, or from about 450 pg/ml to about 500 pg/ml.

In some embodiments, the implant is administered to the subject in the form of a patch. In some embodiments, the implant is administered to the subject subdermally, optionally wherein the implant is inserted beneath a segment of skin on the subject's arm, among other locations (e.g., the subject's abdomen). In some embodiments, the implant is administered onto one, or both, of the subject's ovaries. In some embodiments, the implant is administered into the subject's uterus, optionally wherein the implant is embedded into the subject's uterine lining.

In some embodiments, the implant is administered to the subject at a frequency of no greater than once every 6, 7, 8, 9, 10, 11, or 12 months. In some embodiments, the implant is administered to the subject at a frequency of no greater than once every 24, 36, 48, or 52 weeks. In some embodiments, the implant is administered to the subject at a frequency of no greater than once every one, two, or three years. In some embodiments, a previously administered implant is removed from the subject prior to administration of a subsequent implant.

In another aspect, the disclosure features a kit containing the implant of any one of the above aspects or embodiments of the disclosure, and a package insert. In some embodiments, the package insert instructs a user of the kit to perform the method of any of the above aspects or embodiments of the disclosure.

In another aspect, the disclosure features an implant for ovarian decline, the implant containing:
at least an engineered ovarian support cell;
an extracellular matrix substrate; and
a delivery apparatus.

In some embodiments, the at least an engineered ovarian support cell further comprises an engineered granulosa cell. In some embodiments, the at least an engineered ovarian support cell further comprises an engineered lutein cell. In some embodiments, the at least an engineered ovarian support cell further comprises an engineered theca cell.

In some embodiments, the at least an engineered ovarian support cell is engineered to secrete a hormone in a cyclic dose.

In some embodiments, the extracellular matrix substrate further comprises a collagen polymer matrix. In some embodiments, the collagen polymer matrix is about 100-500 micron particles. In some embodiments, the collagen polymer matrix is about 500-800 micron cubes. In some embodiments, the collagen polymer matrix further comprises a perforated sheet. In some embodiments, the at least an engineered ovarian support cell is embedded within the extracellular matrix substrate. In some embodiments, the delivery apparatus further comprises a subdermal rod. In some embodiments, the implant is formulated as a patch containing a membrane surrounding the delivery apparatus. In some embodiments, the membrane encapsulates the at least an engineered ovarian support cell. In some embodiments, the membrane provides bi-directional flow of a hormone. In some embodiments, the delivery apparatus is removable or reversible.

In another aspect, the disclosure features a method for manufacturing an implant for ovarian decline, the method including:
receiving at least an engineered ovarian support cell;
embedding the at least an engineered ovarian support cell into an extracellular matrix to form a repository; and
depositing the repository within a delivery apparatus.

In another aspect, the disclosure features a method for reversing ovarian decline, the method comprising:
obtaining a baseline hormone level relating to a user; and
selecting an implant for ovarian decline as a function of the baseline hormone level.

In some embodiments, the implant further contains:
at least an engineered ovarian support cell;
an extracellular matrix substrate; and
a delivery apparatus.

In some embodiments, selecting the implant further comprises selecting at least an engineered ovarian support cell as a function of the baseline hormone level.

In some embodiments, selecting the at least an engineered ovarian support cell further comprises selecting a cell quantity.

### BRIEF DESCRIPTION OF THE DRAWINGS

The accompanying drawings are included to illustrate embodiments of the disclosure and further an understanding of its implementations.

FIG. 1 is an exemplary embodiment of an implant for ovarian decline.

FIG. 2 is an exemplary flow diagram of a method for manufacturing an implant for ovarian decline.

FIG. 3 is an exemplary flow diagram of a method reversing ovarian decline.

FIG. 4 is an exemplary flow diagram of a method for administering an implant for ovarian decline;

FIG. 5 is an exemplary embodiment of an ECM for encapsulating an engineered ovarian support cell.

FIG. 6 is an exemplary diagram of a cyclic dosing schedule.

FIG. 7 an exemplary graph illustrating production rates of estradiol from engineered ovarian support cells.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the terms “about” or “approximately” refer to a value that is within 10% (10%, 9%, 8%, 7%, 6%, 5%, 4%, 3%, 2%, 1%, or less) above or below the value being described. For instance, the phrase “about 50 mg” refers to a value between and including 45 mg and 55 mg.

As used herein, the term “assisted reproductive technology” or “ART” refers to a fertility treatment in which one or more female gametocytes (oocytes) or gametes (ova) are manipulated ex vivo so as to promote the formation of an embryo that can, in turn, be implanted into a subject in an effort to achieve pregnancy. For example, in some embodiments, an oocyte retrieved from a subject undergoing an ART procedure may be matured in vitro using, e.g., co-culturing methodologies described herein. In some embodiments, upon the formation of a mature oocyte (ovum), the ovum may be treated with one or more sperm cells so as to promote the formation of a zygote and, ultimately, an embryo. The embryo may then be transferred to the uterus of a female subject, for instance, using the compositions and methods in the art. Exemplary ART procedures include in vitro fertilization (IVF) and intracytoplasmic sperm injection (ICSI) techniques described herein and known in the art.

As used herein, the terms “subject” refers to an organism that receives treatment for a particular disease or condition as described herein. Examples of subjects and subjects include mammals, such as humans (e.g., a female human), receiving treatment for diseases or conditions that correspond to a reduced ovarian reserve or release of immature oocytes.

As used herein, the term “controlled ovarian hyperstimulation” refers to a procedure in which ovulation is induced in a subject, such as a human subject, prior to oocyte or ovum retrieval for use in embryo formation, for instance, by in vitro fertilization (IVF) or intracytoplasmic sperm injection (ICSI).

Controlled ovarian hyperstimulation procedures may involve administration of follicle-stimulating hormone (FSH), human chorionic gonadotropin (hCG), and/or a gonadotropin-releasing hormone (GnRH) antagonist to the subject so as to promote follicular maturation. Controlled ovarian hyperstimulation methods are known in the art and are described herein as they pertain to methods for inducing follicular maturation and ovulation in conjunction with assisted reproductive technology.

As used herein, the term “derived from” in the context of a cell derived from a subject refers to a cell, such as a mammalian ovum, that is either isolated from the subject or obtained from expansion, division, maturation, or manipulation (e.g., ex vivo expansion, division, maturation, or manipulation) of one or more cells isolated from the subject. For instance, an ovum is “derived from” a subject or an oocyte as described herein if the ovum is directly isolated from the subject or obtained from the maturation of an oocyte isolated from the subject, such as an oocyte isolated from the subject from about 1 day to about 5 days following the subject receiving ovarian hyperstimulation procedures (e.g., an oocyte isolated from the subject from about 2 days to about 4 days following ovarian hyperstimulation procedures).

As used herein, the term “dose” refers to the quantity of a therapeutic agent, such as a follicle stimulating agent described herein, that is administered to a subject for the treatment of a disorder or condition, such as to enhance oocyte maturation and/or release and promote retrieval and ex vivo maturation of viable oocytes. A therapeutic agent as described herein may be administered in a single dose or in multiple doses. In each case, the therapeutic agent may be administered using one or more unit dosage forms of the therapeutic agent. For instance, a single dose of 100 mg of a therapeutic agent may be administered using, e.g., two 50 mg unit dosage forms of the therapeutic agent. Similarly, a single dose of 300 mg of a therapeutic agent may be administered using, e.g., six 50 mg unit dosage forms of the therapeutic agent or two 50 mg unit dosage forms of the therapeutic agent and one 200 mg unit dosage form of the therapeutic agent, among other combinations. Similarly, a single dose of 900 mg of a therapeutic agent may be administered using, e.g., six 50 mg unit dosage forms of the therapeutic agent and three 200 mg unit dosage forms of the therapeutic agent or ten 50 mg unit dosage form of the therapeutic agent and two 200 mg unit dosage forms of the therapeutic agent, among other combinations.

As used herein, the term “follicle-stimulating hormone” (FSH) refers to a biologically active heterodimeric human fertility hormone capable of inducing ovulation in a subject. FSH may be purified from post-menopausal human urine or produced as a recombinant protein product. Exemplary recombinant FSH products include follitropin alfa (GONAL-FR, Merck Serono/EMD Serono) and follitropin beta (PUREGON™/FOLLISTIM®, MSD/Scherig-Plough).

As used herein, the term “human chorionic gonadotropin” (hCG) refers to the polypeptide hormone that interacts with the luteinizing hormone chorionic gonadotropin receptor (LHCGR) to induce follicle maturation and ovulation. hCG may be purified from the urine of pregnant women or produced as a recombinant protein product. Exemplary recombinant hCG products include choriogonadotropin alfa (OVIDREL®, Merck Serono/EMD Serono).

As used herein, the term “in vitro fertilization” (IVF) refers to a process in which an ovum, such as a human ovum, is contacted ex vivo with one or more sperm cells so as to promote fertilization of the ovum and zygote formation. The ovum can be derived from a subject, such as a human subject, undergoing various ARTs known in the art. For instance, one or more oocytes may be obtained from the subject following injection of follicular maturation stimulating agents for controlled ovarian hyperstimulation procedures, e.g., from about 1 day to about 5 days prior after injection of said agents (such as from about one day to about 4 days after injection of follicular maturation stimulating agents to the subject). The ovum may also be retrieved directly from the subject, for instance, by transvaginal ovum retrieval procedures known in the art.

As used herein, the term “intracytoplasmic sperm injection” (ICSI) refers to a process in which a sperm cell is injected directly into an ovum, such as a human ovum, so as to promote fertilization of the ovum and zygote formation. The sperm cell may be injected into the ovum, for instance, by piercing the oolemma with a microinjector so as to deliver the sperm cell directly to the cytoplasm of the ovum. ICSI procedures useful in conjunction with the compositions and methods described herein are known in the art and are described, for instance, in WO 2013/158658, WO 2008/051620, and WO 2000/009674, among others, the disclosures of which are incorporated herein by reference as they pertain to compositions and methods for performing intracytoplasmic sperm injection.

As used herein, the terms “ovum” and “oocyte” refer to a haploid female reproductive cell or gamete. In the context of assisted reproductive technology as described herein, ova may be produced ex vivo by maturation of one or more oocytes isolated from a subject undergoing ART. Ova may also be isolated directly from the subject, for example, by transvaginal ovum retrieval methods described herein or known in the art. Ovum or oocyte as used in this disclosure may refer to a plurality of oocytes. An oocyte may be in complex with surrounding cells such as a cumulus-oocyte complex (COC).

As used herein, the terms “mature ova” and “mature oocyte” refer to one or more ovum or oocyte in metaphase II (MII)-stage of meiosis and typically has morphological or structural features consistent with metaphase Il, such as a polar body and other features described herein.

As used herein, the terms “immature ovum” and “immature oocyte” refer to one or more ovum or oocyte that has not reached MII stage of meiosis. In some embodiments, an immature oocyte may be an oocyte including germinal vesicle (GV)-stage and/or metaphase I (MI)-stage oocytes as determined by morphological features and/or other indications known in the art.

As used herein, the term “oocyte maturation” refers to the process by which an immature oocyte developmentally transitions to a mature oocyte. Oocyte maturation occurs as immature oocytes undergo cell signaling events incurred by external and internal stimuli. External stimuli may be produced by neighboring cells or supporting cells described herein. Oocyte maturation may occur prior to the release of an oocyte and retrieval from a subject. Oocyte maturation may occur following administration of an implant as described herein.

As used herein, an “ovarian support cell” (OSC) or “support cell” refers to one or more cells that promotes maturation of one or more oocytes. An OSC may be an ovarian granulosa cell (e.g., a type of granulosa cell described herein), an ovarian lutein cell (e.g., a type of lutein cell described herein), and/or an ovarian theca cell (e.g., a type of theca cell described herein). An OSC may form a cumulus-oocyte complex (COC) with an oocyte. An OSC may be generated from an exogenous source, such as from induced pluripotent stem cells (iPSCs), e.g., human induced pluripotent stem cells (hiPSCs), as described herein. An OSC may be applied to a retrieved oocyte using in vitro cell culture methods and compositions described herein. An OSC may be a mixture of two or more cell types. An OSC may be a mixture of granulosa cells, lutein cells, and theca cells, such that the mixture is approximately a 1:1:1 population of granulosa cells, lutein cells, and theca cells. An OSC may be a mixture of granulosa cells, lutein cells, and theca cells such that one cell type is in higher relative abundance compared to one or more cell types such that the mixture is approximately a 2:1:1 population, a 2:2:1 population, a 3:1:1 population, a 3:2:1 population, a 3:3:1 population, a 4:1:1 population, a 4:3:1 population, a 4:2:1 population, a 4:4:1 population, a 5:1:1 population, a 5:4:1 population, a 5:3:1 population, a 5:2:1 population, a 5:5:1 population, among other possible population distributions. An OSC may be a mixture of granulosa cells, lutein cells, and theca cells such that one cell type is more abundant in the mixture (e.g., 90% granulosa cells, 5% lutein cells, and 5% theca cells; 80% granulosa cells, 10% lutein cells, and 10% theca cells; 70% granulosa cells, 20% lutein cells, and 10% theca cells; 60% granulosa cells, 20% lutein cells, and 20% theca cells; 50% granulosa cells, 20% lutein cells, and 30% theca cells; 40% granulosa cells, 30% lutein cells, and 30% theca cells; 30% granulosa cells, 20% lutein cells, and 50% theca cells; 20% granulosa cells, 50% lutein cells, and 30% theca cells; 10% granulosa cells, 70% lutein cells, and 20% theca cells, among other possible combinations of cell distributions). In some embodiments, an OSC may be a mixture of granulosa cells, lutein cells, and theca cells in combination with one or more additional cell types.

As used herein, an “engineered ovarian support cell” or “engineered OSC” is a reproductive cell originating in an ovary that is genetically engineered in vitro. An ovarian support cell may originate from hIPSCs or ovarian cells and engineered as described herein. An engineered ovarian support cell may be a granulosa cell, a lutein cell, and/or a theca cell. An engineered ovarian support cell may be manipulated using methods known in the art to express or overexpress one or more proteins such as transcription factors, enzymes, or secreted hormones. For instance, an engineered ovarian support cell may include an engineered granulosa cell configured to express and/or overexpress transcription factor RUNX1. As another example, an engineered ovarian support cell may be engineered to express or overexpress quantities of forms of estrogen, e.g., estrone (E1), estradiol (E2), estriol (E3), estetrol (E4), and/or a combination thereof. An engineered ovarian support cell may overexpress a particular protein and/or transcription factor if a particular protein and/or transcription factor level is detectable at a higher reference range. For example, an engineered ovarian support cell may overexpress a particular protein if the protein is detectable at a level that is 5% higher than the level of the protein expressed from an endogenous naturally occurring polynucleotide encoding the protein.

As used herein, an “ovarian granulosa cell” or a “granulosa cell” is a cumulus cell surrounding the oocyte to ensure healthy oocyte and subsequent embryo development. An ovarian granulosa cell may form a COC with an oocyte. An ovarian granulosa cell may express markers consistent with a granulosa subtype such as FOXL2, CD82 and/or follicle-stimulating hormone receptor (FSHR), which can be detected by methods known in the art. An ovarian granulosa cell may be a steroidogenic granulosa cell. An ovarian granulosa cell may be produced from differentiated hiPSCs or otherwise engineered as described herein.

As used herein, a “steroidogenic granulosa cell” is a granulosa cell that may produce one or more steroids such as estradiol, progesterone, or a combination thereof. One or more steroids may be produced in response to hormonal stimulation, such as by FSH, androstenedione, or a combination thereof. One or more steroids may be secreted.

As used herein, a “lutein cell” is a cell of the corpus luteum and secretes progesterone. A lutein cell may be produced from differentiated hiPSCs or otherwise engineered as described herein.

As used herein, an “ovarian theca cell” or a “theca cell” is one or more endocrine cells associated with ovarian follicles and produces one or more androgens and progesterone in the pre-ovulatory large follicles. A theca cell may be produced from differentiated hiPSCs or otherwise engineered as described herein.

As used herein, an “androgen” is any natural or synthetic steroid hormone that regulates the development and maintenance of male characteristics in vertebrates by binding to androgen receptors and are the precursors to estrogens in both males and females. Androgens are natural hormones and are also used as medications in hormone replacement therapy. An androgen may include, e.g., testosterone, dehydroepiandrosterone (DHEA), androstenedione, dehydroepiandrosterone sulfate (DHEA-S), and/or dihydrotesterone.

As used herein, “progesterone” is a steroid hormone belonging to a class of hormones called progestogens. Physiologically, progesterone prepares the endometrium for the potential of pregnancy after ovulation by triggering the uterine lining to thicken to accept a fertilized egg. Further, progesterone prohibits muscle contractions in the uterus that would cause the body to reject an egg. Low levels of progesterone, in relation to ovarian decline, may cause abnormal menstrual cycles or infertility because the progesterone does not induce an environment conducive for the growth and maturation of a conceived egg.

As used herein, “estrogen” is a category of sex hormone responsible for the development and regulation of the female reproductive system and secondary sex characteristics. Estrogens are required for female fertility and are involved in the maturation and maintenance of the vagina, uterus, ovaries and ovarian function, and ovarian follicles.

As used herein, the terms “extracellular matrix” and “extracellular substrate” are used, interchangeably, to refer to a substrate containing one or more extracellular macromolecules and/or minerals in a network that functions to provide biomechanical structure and/or biochemical support (e.g., signaling cues) to one or more adherent cells.

As used herein, the term “biological sample” or “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, hair, oocyte, ovum, and/or cells isolated from a subject.

As used herein, the term “oral contraceptive treatment,” “oral contraception,” “contraception,” or “birth control pill” refers to a hormonal method of treatment typically used to prevent pregnancy. Oral contraceptive treatment may block the release of oocytes from the ovaries and may contain hormones including estrogen and progestin.

As used herein, the term “ovarian decline” refers to a decline in ovarian function in a subject that typically results in diminished reproductive potential. Ovarian decline may manifest as a reduced ovarian reserve. Ovarian decline naturally occurs with age but may be due to genetic or medical conditions such as, e.g., primary ovarian insufficiency (POI), polycystic ovarian syndrome (PCOS), premature menopause, endometriosis, uterine fibroids, gynecological cancer, interstitial cystitis, pelvic inflammatory disease (PID), vaginitis, cervical dysplasia, pelvic floor prolapse, interstitial cystitis, among other nonlimiting conditions.

As used herein, the term “ovarian reserve” refers to the number of oocytes in a subject's ovaries and the quality of said oocytes. The ovarian reserve naturally declines with age and/or medical conditions described herein. Subjects with a diminished ovarian reserve may seek IVF or other ARTs to achieve a successful pregnancy. Levels of anti-Mullerian hormone (AMH), as described herein, may be indicative of a subject's ovarian reserve.

As used herein, a “cyclic dose” is a hormone dose administered to a subject in need thereof in a cyclical pattern as described herein to effectively mimic the natural fluctuations of the amount or types of hormones secreted in the body in response to stimuli. A cyclic dose is unlike a continuous dose, in which a fixed dose of a substance is administered to a subject but is not tailored to mimic natural fluctuations of endogenous hormone delivery.

As used herein, a “cyclical pattern” is the level and type of hormone secreted in response to a subject's brain signaling for production and release of a hormone. The levels and specific secretions fluctuate over time in response to extracellular signals, e.g., from the brain or cellular environment.

As used herein, an “administration site” or implantation site” refers to the site for the application of the implant to the subject in need thereof.

As used herein, the terms “follicular triggering agent” or “triggering agent” refer to a chemical or biological composition that stimulates release of oocytes from the ovaries during ovulation. Follicular triggering agents may include hormones such as human chorionic gonadotropin and follicle-stimulating hormone.

As used herein, the term “induced pluripotent stem cells” (iPSCs) refer to artificial stem cells that derive from reprogrammed and otherwise manipulated harvested somatic cells. iPSCs may differentiate into other cell types including ovarian support cells or granulosa cells via methods known in the art and methods described herein. iPSCs may be humans (hiPSCs) or iPSCs from, e.g., other mammalian sources.

As used herein, the term “IU” refers to international units, which are the units of the internationally accepted measurement of the biological, chemical, or catalytic activity of a particular biological molecule, macromolecule, or chemical compound.

As used herein, the term “cell culture” refers to laboratory methods that enable in vitro cell proliferation and/or cultivation of a population of cells (e.g., a population of ovarian support cells described herein).

As used herein, the term “co-culture” refers to a type of cell culture method in which more than one cell type or cell populations are cultivated with some degree of contact between them. In a typical co-culture system, two or more cell types may share artificial growth medium.

As used herein, the terms “adherent co-culture systems” or “adherent cell culture” refer to a cell culture arrangement by which cells are attached to a surface for proper growth and proliferation.

As used herein, the terms “suspension co-culture systems” or “suspension cell culture” refer to a cell culture arrangement by which cells are cultivated via dispersion in a liquid medium for proper growth and proliferation.

As used herein, unless otherwise stated, all described concentrations or levels of hormones refer to serum concentrations or levels.

### DETAILED DESCRIPTION

Described herein are compositions and methods for use in assisted reproductive technology (ART) and hormone replacement therapy (HRT). For example, the compositions and methods described herein are directed to implants (e.g., ovarian implants) comprising ovarian support cells that are capable of secreting one or more hormones that promote endogenous oocyte maturation, ovulation, and/or the creation of a favorable endometrial environment for embryo implantation. Examples of hormones that may be secreted by the compositions of the disclosure include estradiol, progesterone, and androstenedione.

Without being limited by mechanism, this hormone secretion activity represents one way in which the compositions and methods of the disclosure may recapitulate certain ovarian functions in vivo, thereby treating, ameliorating, or altogether reversing ovarian decline in subjects that have reduced ovarian activity due to a natural, timewise reduction in the ovarian reserve (e.g., due to menopause or other condition described herein). The compositions and methods of the disclosure may also be used to treat subjects having diseases or conditions that result in impaired ovarian function (e.g., primary ovarian insufficiency (POI), polycystic ovarian syndrome (PCOS), premature menopause, endometriosis, uterine fibroids, gynecological cancer, interstitial cystitis, pelvic inflammatory disease (PID), vaginitis, cervical dysplasia, uterine fibroids, pelvic floor prolapse, and interstitial cystitis, among other indications described herein).

Accordingly, the compositions and methods described herein may supplement or replace the estrogen-, progesterone-, and/or androstenedione-secreting functions of a healthy ovary. The compositions and methods of the disclosure provide the benefit of being responsive to the same endogenous gonadal hormones that stimulate estrogen, progesterone, and/or androstenedione under standard, pre-menopausal conditions-namely, gonadotropin-releasing hormone (GnRH), luteinizing hormone (LH), and follicle-stimulating hormone (FSH). Since these gonadal hormones are secreted by the hypothalamus and pituitary on a cyclic basis (e.g., over the course of a typical 28-32 day menstrual cycle), the implants of the disclosure are capable of secreting estrogen, progesterone, and/or androstenedione in a cyclic manner that approximates the standard, pre-menopausal timeline observed in subjects having healthy ovarian function.

Advantageously, the compositions and methods of the disclosure do not require genetic modification in order to achieve the cyclic estrogen, progesterone, and/or androstenedione secretion characteristic of a healthy, pre-menopausal subject. Instead, the compositions and methods described herein leverage the natural, periodic secretion of hormones on the hypothalamic-pituitary-gonadal (HPG) axis in order to supplement or altogether recapitulate ovarian activity. By responding to the body's natural HPG axis fluctuations, OSC implants herein offer a new and improved form of personalized treatment to a wide variety of subjects seeking to supplement ovarian function or ameliorate ovarian decline. The compositions and method of the disclosure may reduce the need for continuous medical interventions, such as hormonal injection therapies, thereby reducing certain health risks that are associated with traditional hormone replacement therapy. Chief among these risks is the risk of cancer that is associated with continuous exposure to high levels of exogenously injected hormones.

The following sections describe, in further detail, the structural and functional characteristics of OSC implants of the disclosure, as well as how these implants may be used to treat, ameliorate, or reverse ovarian decline or dysfunction.

### A. Components of Ovarian Support Cell (OSC) Implants

The disclosed ovarian support cell (OSC) implants comprise a matrix of cells and proteinaceous components that supplement the levels of ovarian hormones secreted in the bloodstream of a healthy, pre-menopausal subject. The OSC implants secrete hormones such as estrogen (e.g., estradiol), progesterone, and androstenedione into the bloodstream in response to endogenous, temporal (e.g., cyclic) signals released by the hypothalamus and pituitary gland. An OSC implant may contain one or more ovarian granulosa cells, lutein cells, and/or theca cells. Such implants may also contain one or more substrates, as is described in further detail below.

### Cellular Components

An OSC implant may include ovarian granulosa, ovarian lutein cells, and/or ovarian theca cells, optionally in combination with one or more additional cell types. The distribution of cells in an OSC implant may depend on a subject's particular hormonal, biological, or physiological needs, which, if necessary, may be determined by a skilled practitioner (e.g., a physician, an OB/GYN, a nurse practitioner, or another skilled professional). In some embodiments, an OSC implant comprises an equal or relatively equal distribution of granulosa, lutein cells, and ovarian theca cells. In some embodiments, an OSC implant comprises a distribution of cells in which one cell type is more abundant than the other cell types in the population. In other embodiments, an OSC implant comprises a distribution of cells in which each cell type is represented at a different relative abundance such that no two cell types have an equivalent relative distribution.

An OSC implant may include a predetermined number of granulosa cells for implantation. In some embodiments, granulosa cells in an OSC are implanted at a range between 1×10⁶ and 1×10⁷ cells (e.g., between 1×10⁶ and 2×10⁶, between 2×10⁶ and 3×10⁶, between 3×10⁶ and 4×10⁶, between 4×10⁶ and 5×10⁶, between 5×10⁶ and 6×10⁶, between 6×10⁶ and 7×10⁶, between 7×10⁶ and 8×10⁶, between 8×10⁶ and 9×10⁶, or between 9×10⁶ and 1×10⁷; e.g., about 1×10⁶ cells, about 2×10⁶ cells, about 3×10⁶ cells, about 4×10⁶ cells, about 5×10⁶ cells, about 6×10⁶ cells, about 7×10⁶ cells, about 8×10⁶ cells, about 9×10⁶ cells, about 1×10⁷ cells). A granulosa cell is a cumulus cell surrounding the oocyte to ensure healthy oocyte and subsequent embryo development. An ovarian granulosa cell may express markers consistent with a granulosa subtype such as FOXL2, CD82 and/or follicle-stimulating hormone receptor (FSHR), which can be detected by methods known in the art. An ovarian granulosa cell may express one or more transcription factors selected from FOXL2, NR5A1, GATA4, RUNX1, RUNX2, or a combination thereof, at higher levels compared to an accepted reference ovarian cell or other cell type known in the art. An ovarian granulosa cell may be a steroidogenic granulosa cell. An ovarian granulosa cell in an OSC implant may secrete levels of E2 consistent with various phases of the menstrual cycle in healthy, pre-menopausal ovaries as described in detail in the following section. An ovarian granulosa cell may derive from hiPSCs as described herein.

An OSC implant may include a predetermined number of ovarian lutein cells for implantation. In some embodiments, lutein cells in an OSC are implanted at a range between 1×10⁶ and 1×10⁷ cells (e.g., between 1×10⁶ and 2×10⁶, between 2×10⁶ and 3×10⁶, between 3×10⁶ and 4×10⁶, between 4×10⁶ and 5×10⁶, between 5×10⁶ and 6×10⁶, between 6×10⁶ and 7×10⁶, between 7×10⁶ and 8×10⁶, between 8×10⁶ and 9×10⁶, or between 9×10⁶ and 1×10⁷; e.g., about 1×10⁶ cells, about 2×10⁶ cells, about 3×10⁶ cells, about 4×10⁶ cells, about 5×10⁶ cells, about 6×10⁶ cells, about 7×10⁶ cells, about 8×10⁶ cells, about 9×10⁶ cells, about 1×10⁷ cells). A lutein cell is a cell of the corpus luteum. In some embodiments, a lutein cell may express one or more of the following markers consistent with the lutein cell subtype such as KRT19, CYP19A1, STAR, CYP17A1, PGR, or a combination thereof. A lutein cell secretes progesterone during the luteal phase of the menstrual cycle at levels described in detail in the following section. An ovarian lutein cell may derive from hiPSCs as described herein.

An OSC implant may include a predetermined number of ovarian theca cells for implantation. In some embodiments, theca cells in an OSC are implanted at a range between 1×10⁶ and 1×10⁷ cells (e.g., between 1×10⁶ and 2×10⁶, between 2×10⁶ and 3×10⁶, between 3×10⁶ and 4×10⁶, between 4×10⁶ and 5×10⁶, between 5×10⁶ and 6×10⁶, between 6×10⁶ and 7×10⁶, between 7×10⁶ and 8×10⁶, between 8×10⁶ and 9×10⁶, or between 9×10⁶ and 1×10⁷; e.g., about 1×10⁶ cells, about 2×10⁶ cells, about 3×10⁶ cells, about 4×10⁶ cells, about 5×10⁶ cells, about 6×10⁶ cells, about 7×10⁶ cells, about 8×10⁶ cells, about 9×10⁶ cells, about 1×10⁷ cells). An ovarian theca cell may express higher levels of transcription factors NR2F2 and/or GATA4 compared to an accepted reference ovarian cell or other cell type known in the art. In the ovary, theca cells convert cholesterol to androstenedione, which is the substrate for estradiol production in granulosa cells. An ovarian theca cell in an OSC implant may secrete androstenedione during the luteal phase and/or ovulation described below.

In some embodiments, a quantity of OSCs is selected that will provide the subject with a serum concentration of one or more estrogens (e.g., estradiol), progestogens (e.g., progesterone), and/or androgens (e.g., androstenedione) recited in the section entitled “Cyclic hormone release,” below.

### OSCs From hiPSCs

Specialized granulosa cells, lutein cells, or theca cells utilized in the methods described herein may be created from hiPSCs using transcription factor (TF)-directed protocols. In some embodiments, hiPSCs may be transformed with any one or more plasmids encoding one or more transcription factors. In some embodiments, hiPSCs may be transformed via electroporation, liposome-mediated transformation, viral-mediated gene transfer, among other cell transformation methodologies known in the art. In some embodiments, gene expression of desired transcription factors may be induced in a doxycycline-dependent manner. In some embodiments, transcription factors are constitutively expressed. In some embodiments, a plasmid or expression vector used for reprogramming hiPSCs may have a reporter gene such as a fluorescent protein. In some embodiments, hiPSCs may differentiate into stroma cells with induced expression of transcription factors including GATA4, FOXL2, or a combination thereof. In some embodiments, hiPSCs may differentiate into granulosa with induced expression of transcription factors including FOXL2, NR5A1, GATA4, RUNX1, RUNX2, or a combination thereof. In addition to a combination of one or more transcription factors of FOXL2, NR5A1, GATA4, RUNX1, and/or RUNX2, hiPSCs may differentiate into granulosa via expression of KLF2, TCF21, NR2F2, or a combination thereof. In another embodiment, an hiPSC may be engineered to include an open reading frame encoding one or more proteins selected from zinc finger protein 281 (ZNF281), LIM homeobox 8 (LHX8), spermatogenesis and oogenesis specific basic helix-loop-helix 1 (SOHLH1), distal-less homeobox 5 (DLX5), hematopoietically-expressed homeobox protein (HHEX), folliculogenesis specific BHLH transcription factor (FIGLA), or a combination thereof.

In some embodiments, hiPSCs may differentiate into lutein cells with induced expression of KRT19, CYP19A1, STAR, CYP17A1, PGR, or a combination thereof.

In some embodiments, hiPSCs may differentiate into theca cells with induced expression of NR2F2 and/or GATA4.

Reprogramming of hiPSCs to granulosa, lutein cells, or theca cells may be determined by genotyping methods known in the art. Reprogramming of hiPSCs to granulosa may be determined by protein expression using any one or more methods known in the art. Differentiation of hiPSCs to granulosa cells may be determined by relative expression of biomarkers typical of a granulosa cell type including AMHR2, CD82, FOXL2, FSHR, IGFBP7, KRT19, STAR, WNT4, or a combination thereof among other granulosa cell biomarkers known in the art. In some embodiments, reprogramming of hiPSCs to granulosa may be determined by production of growth factors and/or hormones including estradiol and progesterone that may adequately support in vitro maturation of retrieved oocyte via paracrine and juxtacrine cell signaling. In some embodiments, the resulting granulosa cells produce estradiol upon stimulation of androstenedione and FSH or forskolin. In some embodiments, the granulosa cells described herein may be produced in multiple batches. In some embodiments, the granulosa cells may be frozen and thawed prior to co-culture methods. In some embodiments, the granulosa cells were freshly differentiated prior to in vitro maturation method. In some embodiments, the granulosa cells may be seeded and equilibrated for 2-8 hours (e.g., 2-3 hours, 2-4 hours, 3-4 hours, 4-6 hours, 5-7 hours, 6-8 hours; e.g., 2 hours, 3 hours, 4 hours, 5 hours, 6 hours, 7 hours, 8 hours) before the addition of oocytes for in vitro maturation.

In some embodiments, a subject may donate hiPSCs. hiPSCs donation may follow an oocyte retrieval process as discussed above. A subject participating in hiPSCs donation may be different, or the same, from the subject from which the oocyte was retrieved. In some embodiments, a hiPSC donor may undergo a stimulation protocol as disclosed above.

### Transgenic OSCs for Implantation

Specialized transgenic OSCs may be produced using Clustered Regularly Interspaced Short Palindromic Repeats (CRISPR) technology. “CRISPR” is programmable technology that targets specific stretches of genetic code to edit DNA at precise locations. CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platforms and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of in vitro Transcription (SPRINT). SPRINT can be used to detect a variety of substances, such as metabolites in subject samples or contaminants in environmental samples, with high throughput or with portable point-of-care devices.

### Cell Culture Conditions

OSCs for implantation, such as granulosa cells, lutein cells, and theca cells optionally derived from iPSCs (e.g., hiPSCs) or transgenic cells (as described above), may be provided as a composition further containing a cell culture media (e.g., IVF, IVM, (e.g., MediCult IVM media), or LAG media). The cell culture media may include human serum albumin (e.g., at about 5-15 mg/ml, e.g., 10 mg/ml), FSH (e.g., at about 70-80 mlU/ml, e.g., 75 mlU/ml), hCG (e.g., at about 95-105 mlU/ml, e.g., 100 mlU/ml), Androstenedione (e.g., at about 495-505 ng/ml, e.g., 500 ng/ml), Doxycycline (e.g., 0.5-1.5 μg/ml, e.g., 1 μg/ml) and other compounds such as hyaluronidase and/or dPBS.

### Inactive ingredients

An OSC implant may comprise inactive ingredients, including extracellular matrix components that provide biochemical and biomechanical structure and support for the active cellular components.

In some embodiments, an OSC implant is embedded into an extracellular matrix (ECM) comprising one or more ECM proteins and associated molecules such as binding partners or ions (e.g., divalent ions; e.g., calcium ions and/or magnesium ions). In some embodiments, an OSC implant comprises ECM proteins, protein polymers, and molecules that can be found in connective tissue. In some embodiments, the ECM comprises one or more types of collagen (e.g., fibrillar collagen; e.g., collagen I, II, III, V, XI), one or more epidermal growth factors (EGFs), elastin (e.g., tropoelastin or mature elastin), fibronectin, vitronectin, laminin, among other glycoproteins, cell adhesion proteins, or plant-derived proteins or protein polymers (e.g., alginate). In some embodiments, the ECM comprises polymers ranging from 500 to 800 μm in size (e.g., 500 to 550 μm, 550 to 600 μm, 600 to 650 μm, 650 to 700 μm, 700 to 750 μm, or 750 to 800 μm; e.g., 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 74 μm, 750 μm, 760 μm, 770 μm, 780 μm, 790 μm, or 800 μm). Other embodiments of an ECM of an OSC implant include proteoglycans, heparan sulfate, chondroitin sulfate, keratan sulfate, hyaluronic acid, elastin, dermatan sulfate, extracellular vesicles, nanoparticles, microparticles, proteins, cell adhesion proteins, proteoglycans, carbohydrate polymers, non-proteoglycan polysaccharides, and other forms of substrates one skilled in the art may understand as applicable to embedding and supporting an OSC implant. In some embodiments, one or more ECM components are derived from purified or partially purified animal or plant tissue. In other embodiments, one or more ECM components are derived from in vitro cell culturing conditions.

### B. Cyclic Hormone Release

Cyclic hormone release refers to the physiological phenomenon of natural fluctuations of hormone secretions in the body over a period of time. Upon ovarian decline, the ovarian cells have a reduced capacity of responding to the body's continued cyclic release of hormones. The cyclic hormone release is controlled by the hypothalamus and pituitary gland and results in temporally increased secretion of FSH, LH, and GnRH. Upon administration of an OSC implant, the implant may help to restore the subject's ability to respond to cyclic fluctuations in HPG axis hormones by providing a synchronized, time-dependent release of estrogen, progesterone, and androstenedione in a manner that approximates the cyclic secretion of these hormones by a healthy, pre-menopausal subject.

The OSC may work in tandem with the ovaries to produce sufficient hormone levels that can be measured using methods known in the art. For example, a subject in ovarian decline may still produce estrogen but not at levels sufficient for fertility, and the OSC implant may secrete a dose of estrogen that matches the levels of healthy, pre-menopausal ovaries. In some embodiments, the implant may be the sole source of the hormone. In some embodiments, the implant recapitulates the variable, fluctuating estrogen levels during the different phases of the menstrual and follicular cycles. In some embodiments, the OSC implant secretes estradiol levels that are consistent with the early to mid-follicular phase (i.e., the first week of the menstrual cycle or menses) and range from 20 to 80 pg/ml (e.g., 20 to 30 pg/ml, 30 to 40 pg/ml, 40 to 50 pg/ml, 50 to 60 pg/ml, 60 to 70 pg/ml, or 70 to 80 pg/ml; e.g., 20 pg/ml, 21 pg/ml, 22 pg/ml, 23 pg/ml, 24 pg/ml, 25 pg/ml, 26 pg/ml, 27 pg/ml, 28 pg/ml, 29 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, or 80 pg/ml).

In some embodiments, the OSC implant secretes gradually increasing levels of estradiol consistent with the mid to late follicular phase (i.e., the second week of the menstrual cycle) until the pre-ovulatory phase. In some embodiments, the OSC implant secretes levels of estradiol consistent with the mid to late follicular phase at level between 20 and 200 pg/ml (e.g., between 20 and 50 pg/ml, between 50 and 70 pg/ml, between 70 and 90 pg/ml, between 90 and 110 pg/ml, between 110 and 130 pg/ml, between 130 and 150 pg/ml, between 150 and 170 pg/ml, or between 170 and 200 pg/ml; e.g., 20 pg/ml, 21 pg/ml, 22 pg/ml, 23 pg/ml, 24 pg/ml, 25 pg/ml, 26 pg/ml, 27 pg/ml, 28 pg/ml, 29 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 90 pg/ml, 100 pg/ml, 110 pg/ml, 120 pg/ml, 130 pg/ml, 140 pg/ml, 150 pg/ml, 160 pg/ml, 170 pg/ml, 180 pg/ml, 190 pg/ml, or 200 pg/ml).

At the time of pre-ovulation (a period of about 24 to 48 hours), estradiol levels may rise. In some embodiments, the OSC implant secretes estradiol levels consistent with the pre-ovulation phase at a level ranging from 130 to 200 pg/ml (e.g., 130 to 140 pg/ml, 140 to 150 pg/ml, 150 to 160 pg/ml, 160 to 170 pg/ml, 170 to 180 pg/ml, 180 to 190 pg/ml, 190 to 200 pg/ml, or above 200 pg/ml; e.g., 130 pg/ml, 131 pg/ml, 132 pg/ml, 133 pg/ml, 134 pg/ml, 135 pg/ml, 136 pg/ml, 137 pg/ml, 138 pg/ml, 139 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, 200 pg/ml, or higher). In some embodiments, the OSC implant secretes estradiol levels consistent with higher circulating levels of estradiol observed in some subjects during the pre-ovulation phase, such that the OSC implant secretes levels of estradiol between 300 and 400 pg/ml (e.g., 300 to 310 pg/ml, 310 to 320 pg/ml, 320 to 330 pg/ml, 330 to 340 pg/ml, 340 to 350 pg/ml, 350 to 360 pg/ml, 360 to 370 pg/ml, 370 to 380 pg/ml, 380 to 390 pg/ml, 390 to 400 pg/ml, or above 400 pg/ml; e.g., 300 pg/ml, 301 pg/ml, 302 pg/ml, 303 pg/ml, 304 pg/ml, 305 pg/ml, 306 pg/ml, 307 pg/ml, 308 pg/ml, 309 pg/ml, 310 pg/ml, 315 pg/ml, 320 pg/ml, 325 pg/ml, 330 pg/ml, 335 pg/ml, 340 pg/ml, 345 pg/ml, 350 pg/ml, 355 pg/ml, 360 pg/ml, 365 pg/ml, 370 pg/ml, 375 pg/ml, 380 pg/ml, 385 pg/ml, 390 pg/ml, 395 pg/ml, 400 pg/ml, or higher).

At the time of ovulation, concentrations of secreted estradiol may surge. In some embodiments, the OSC implant secretes estradiol levels consistent with an ovulatory surge between 250 and 500 pg/ml (e.g., between 250 and 275 pg/ml, between 275 and 300 pg/ml, between 300 and 325 pg/ml, between 325 and 350 pg/ml, between 350 and 375 pg/ml, between 375 and 400 pg/ml, between 400 and 425 pg/ml, between 425 and 450 pg/ml, between 450 and 475 pg/ml, between 475 and 500 pg/ml, or higher; e.g., 250 pg/ml, 260 pg/ml, 270 pg/ml, 280 pg/ml, 290 pg/ml, 300 pg/ml, 310 pg/ml, 320 pg/ml, 330 pg/ml, 340 pg/ml, 350 pg/ml, 360 pg/ml, 370 pg/ml, 380 pg/ml, 390 pg/ml, 400 pg/ml, 410 pg/ml, 420 pg/ml, 430 pg/ml, 440 pg/ml, 450 pg/ml, 460 pg/ml, 470 pg/ml, 480 pg/ml, 490 pg/ml, 500 pg/ml, or higher).

Following ovulation (or mid-cycle) and during the latter half of the menstrual cycle or the luteal phase, estradiol levels plateau and fluctuate during the early and mid-luteal phase. In some embodiments, the OSC implant secretes estradiol levels consistent with the early and mid-luteal phase at a range between 100 and 150 pg/ml (e.g., 100 to 110 pg/ml, 110 to 120 pg/ml, 120 to 130 pg/ml, 130 to 140 pg/ml, or 140 to 150 pg/ml; e.g., 100 pg/ml, 101 pg/ml, 102 pg/ml, 103 pg/ml, 104 pg/ml, 105 pg/ml, 106 pg/ml, 107 pg/ml, 108 pg/ml, 109 pg/ml, 110 pg/ml, 115 pg/ml, 120 pg/ml, 125 pg/ml, 130 pg/ml, 135 pg/ml, 140 pg/ml, 145 pg/ml, or 150 pg/ml). In other embodiments, the OSC implant secretes levels of estradiol consistent with that of the late luteal phase (i.e., a few days before menstruation or menses), such that the secreted level of estradiol is between 60 and 200 pg/ml (e.g., 60 and 80 pg/ml, between 80 and 100 pg/ml, between 100 and 120 pg/ml, between 120 and 140 pg/ml, between 140 and 160 pg/ml, between 160 and 180 pg/ml, between 180 and 200 pg/ml; e.g., 60 pg/ml, 61 pg/ml, 62 pg/ml, 63 pg/ml, 64 pg/ml, 65 pg/ml, 66 pg/ml, 67 pg/ml, 68 pg/ml, 69 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, or 200 pg/ml).

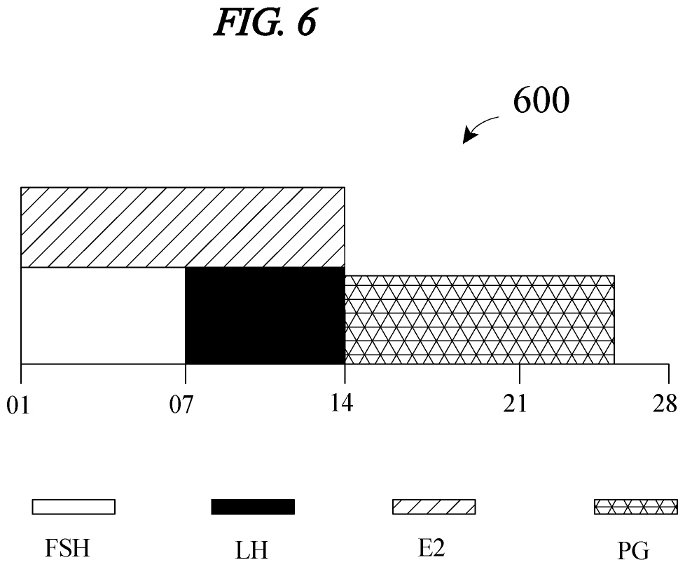

Progesterone levels are relatively low during the pre-ovulatory phase of the menstrual cycle, rise after ovulation, and are elevated during the luteal phase, as shown in FIG. 6. Progesterone levels tend to be less than 2 ng/ml prior to ovulation and greater than 5 ng/ml after ovulation. In some embodiments, an OSC implant secretes progesterone at levels consistent with a pre-ovulatory phase such that an OSC secretes progesterone at levels less than 2 ng/ml (e.g., less than 0.1 ng/ml, between 0.1 and 0.5 ng/ml, between 0.5 and 1 ng/ml, between 1.0 and 1.5 ng/ml, or between 1.5 and 1.9 ng/ml; e.g., 0.1 ng/ml, 0.2 ng/ml, 0.3 ng/ml, 0.4 ng/ml, 0.5 ng/ml, 0.6 ng/ml, 0.7 ng/ml, 0.8 ng/ml, 0.9 ng/ml, 1.0 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, or 1.9 ng/ml). In other embodiments, an OSC implant secretes progesterone at levels consistent with post-ovulation phase at a range greater than 5 ng/ml (e.g., greater than 5 ng/ml, greater than 6 ng/ml, greater than 7 ng/ml, greater than 8 ng/ml, greater than 9 ng/ml, greater than 10 ng/ml, or higher). If pregnancy occurs, human chorionic gonadotropin is released, maintaining the corpus luteum and allowing it to maintain levels of progesterone. Between 7 and 9 weeks, the placenta begins to produce progesterone in place of the corpus luteum in a process called the luteal-placental shift.

Androstenedione levels are higher during the mid-follicular and luteal phases compared to the early follicular phase. In some embodiments, an OSC implant secrets androstenedione at levels between 1 and 3 ng/ml (e.g, between 1 and 1.5 ng/ml, between 1.5 and 2 ng/ml, between 2 and 2.5 ng/ml, or between 2.5 and 3 ng/ml; e.g., 1 ng/ml, 1.5 ng/ml, 2 ng/ml, 2.5 ng/ml, or 3 ng/ml).

LH levels are normally low during childhood and, in women, are high after menopause. In some embodiments, the subject endogenously secretes LH consistent with levels observed during reproductive years, such as levels between 1 and 20 IU/L (e.g., between 1 and 5 IU/L, between 5 and 10 IU/L, between 10 and 15 IU/L, between 15 and 20 IU/L; e.g., about 1 IU/L, about 2 IU/L, about 3 IU/L, about 4 IU/L, about 5 IU/L, about 6 IU/L, about 7 IU/L, about 8 IU/L, about 9 IU/L, about 10 IU/L, about 11 IU/L, about 12 IU/L, about 13 IU/L, about 14 IU/L, about 15 IU/L, about 16 IU/L, about 17 IU/L, about 18 IU/L, about 19 IU/L, about 20 IU/L). Physiologic high LH levels are seen during the LH surge and typically last 48 hours.

FSH levels are typically measured in the early follicular phase of the menstrual cycle, day three to five, counted from last menstruation. At this time, the levels of estradiol (E2) and progesterone are at the lowest point of the menstrual cycle. FSH levels in this time is often called basal FSH levels, to distinguish from the increased levels when approaching ovulation. In some embodiments, the subject endogenously secretes levels of FSH consistent with secreted levels before ovulation at a range between 3.8 to 8.8 mlU/ml (e.g., from 3.8 to 4.8 mlU/ml, from 4.8 to 5.8 mlU/ml, from 5.8 to 6.8 mlU/ml, from 7.8 to 8.8. mlU/ml; e.g., 3.8 mlU/ml, 3.9 mlU/ml, 4.0 mlU/ml, 4.1 mlU/ml, 4.2 mlU/ml, 4.3 mlU/ml, 4.4 mlU/ml, 4.5 mlU/ml, 4.6 mlU/ml, 4.7 mlU/ml, 4.8 mlU/ml, 4.9 mlU/ml, 5.0 mlU/ml, 5.1 mlU/ml, 5.2 mlU/ml, 5.3 mlU/ml, 5.4 mlU/ml, 5.5 mlU/ml, 5.6 mlU/ml, 5.7 mlU/ml, 5.8 mlU/ml, 5.9 mlU/ml, 6.0 mlU/ml, 6.1 mlU/ml, 6.2 mlU/ml, 6.3 mlU/ml, 6.4 mlU/ml, 6.5 mlU/ml, 6.6 mlU/ml, 6.7 mlU/ml, or 6.8 mlU/ml). In some embodiments, the subject endogenously secretes FSH at levels consistent with levels secreted after ovulation between 1.8 and 5.1 mlU/ml (e.g. between 1.8 and 3.3 mlU/ml, between 3.3 and 3.8 mlU/ml, between 3.8 and 4.3 mlU/ml, between 4.3 and 4.8 mlU/ml, or between 4.8 and 5.1 mlU/ml; e.g., 1.8 mlU/ml, 1.9 mlU/ml, 2.0 mlU/ml, 2.1 mlU/ml, 2.3 mlU/ml, 2.4 mlU/ml, 2.5 mlU/ml, 2.6 mlU/ml, 2.7 mlU/ml, 2.8 mlU/ml, 3.1 mlU/ml, 3.5 mlU/ml, 3.8 mlU/ml, 4.1 mlU/ml, 4.5 mlU/ml, 4.8 mlU/ml, or 5.1 mlU/ml). In other embodiments, the subject endogenously secretes FSH at levels consistent with levels secreted in the middle of the menstrual cycle or menses at levels between 4.5 and 22.5 mlU/ml (e.g., between 4.5 and 9.5 mlU/ml, between 9.5 and 13.5 mlU/ml, between 13.5 and 18.5 mlU/ml, or between 18.5 and 22.5 mlU/ml; e.g., 4.5 mlU/ml, 5.0 mlU/ml, 5.5 mlU/ml, 6.0 mlU/ml, 6.5 mlU/ml, 7.0 mlU/ml, 7.5 mlU/ml, 8.0 mlU/ml, 8.5 mlU/ml, 9.0 mlU/ml, 9.5 mlU/ml, 10 mlU/ml, 10.5 mlU/ml, 11 mlU/ml, 11.5 mlU/ml, 12.0 mlU/ml, 12.5 mlU/ml, 13.0 mlU/ml, 13.5 mlU/ml, 14.0 mlU/ml, 14.5 mlU/ml, 15.0 mlU/ml, 15.5 mlU/ml, 16.0 mlU/ml, 16.5 mlU/ml, 17.0 mlU/ml, 17.5 mlU/ml, 18.0 mlU/ml, 18.5 mlU/ml, 19.0 mlU/ml, 19.5 mlU/ml, 20.0 mlU/ml, 20.5 mlU/ml, 21.0 mlU/ml, or 21.5 mlU/ml).

Regarding the cyclic dosing schedule, during the first week of subject's menstrual cycle, and/or the follicular phase, the subject may secrete FSH in specific, characteristic amounts. The follicular phase starts on the first day of menstruation and ends with ovulation. Prompted by the hypothalamus, the pituitary gland releases FSH. This hormone stimulates the ovary to produce around five to 20 follicles (tiny nodules or cysts), which bead on the surface. In some embodiments, during the first week of subject's menstrual cycle, and/or the follicular phase, the subject endogenously secretes FSH in an amount of from 1.4 to 9.9 IU/ml (e.g., 1.4 to 1.9 IU/ml, 1.9 to 2.4 IU/ml, 2.4 to 2.9 IU/ml, 2.9 to 3.4 IU/ml, 3.4 to 3.9 IU/ml, 3.9 to 4.5 IU/ml, 4.5 to 4.9 IU/ml, 4.9 to 5.4 IU/ml, 5.4 to 5.9 IU/ml, 5.9 to 6.4 IU/ml, 6.4 to 6.9 IU/ml, 6.9 to 7.4 IU/ml, 7.4 to 7.9 IU/ml, 7.9 to 8.4 IU/ml, 8.4 to 8.9 IU/ml, 8.9 to 9.4 IU/ml, or 9.4 to 9.9 IU/ml; e.g., 1.4 IU/ml, 1.5 IU/ml, 1.6 IU/ml, 1.7 IU/ml, 1.8 IU/ml, 1.9 IU/ml, 2.0 IU/ml, 2.1 IU/ml, 2.2 IU/ml, 2.3 IU/ml, 2.4 IU/ml, 2.5 IU/ml, 2.9 IU/ml, 3.0 IU/ml, 3.5 IU/ml, 3.9 IU/ml, 4.0 IU/ml, 4.5 IU/ml, 4.9 IU/ml, 5.0 IU/ml, 5.5 IU/ml, 5.9 IU/ml, 6.0 IU/ml, 6.5 IU/ml, 6.9 IU/ml, 7.0 IU/ml, 7.5 IU/ml, 7.9 IU/ml, 8.0 IU/ml, 8.5 IU/ml, 8.9 IU/ml, 9.0 IU/ml, 9.5 IU/ml, or 9.9 IU/ml).

In some embodiments, the OSC implant secretes a level of estradiol consistent with the follicular phase of the menstrual cycle at a range between 550 and 2800 pmol/L (e.g., between 550 and 650 pmol/L, between 650 and 750 pmol/L, between 750 and 850 pmol/L, between 850 and 950 pmol/L, between 950 and 1050 pmol/L, between 1050 and 1150 pmol/L, between 1150 and 1250 pmol/L, between 1250 and 1350 pmoL/L, between 1350 and 1450 pmol/L, between 1450 and 1550 pmol/L, between 1550 and 1650 pmol/L, between 1650 and 1750 pmol/L, between 1750 and 1850 pmol/L, between 1850 and 1950 pmol/L, between 1950 and 2050 pmol/L, between 2050 and 2150 pmol/L, between 2150 and 2250 pmol/L, between 2250 and 2350 pmol/L, between 2350 and 2450 pmol/L, between 2450 and 2550 pmol/L, between 2550 and 2650 pmol/L, between 2650 and 2750 pmol/L, or between 2750 and 2800 pmol/L; e.g., 550 pmol/L, 555 pmol/L, 560 pmol/L, 565 pmol/L, 570 pmol/L, 575 pmol/L, 580 pmol/L, 585 pmol/L, 590 pmol/L, 595 pmol/L, 600 pmol/L, 650 pmol/L, 700 pmol/L, 750 pmol/L, 800 pmol/L, 850 pmol/L, 900 pmol/L, 950 pmol/L, 1000 pmol/L, 1050 pmol/L, 1100 pmol/L, 1150 pmol/L, 1200 pmol/L, 1250 pmol/L, 1300 pmol/L, 1350 pmol/L, 1400 pmol/L, 1450 pmol/L, 1500 pmol/L, 1550 pmol/L, 1600 pmol/L, 1650 pmol/L, 1700 pmol/L, 1750 pmol/L, 1800 pmol/L, 1850 pmol/L, 1900 pmol/L, 1950 pmol/L, 2000 pmol/L, 2050 pmol/L, 2100 pmol/L, 2150 pmol/L, 2200 pmol/L, 2250 pmol/L, 2300 pmol/L, 2350 pmol/L, 2400 pmol/L, 2450 pmol/L, 2500 pmol/L, 2550 pmol/L, 2600 pmol/L, 2650 pmol/L, 2700 pmol/L, 2750 pmol/L, or 2800 pmol/L).

During the second week and/or ovulation phase of the menstrual cycle, LH may be secreted by the subject's endogenous pituitary glands. Ovulation is the release of a mature egg from the surface of the ovary. This usually occurs mid-cycle, around two weeks or so before menstruation starts. During the follicular phase, the developing follicle causes a rise in the level of estrogen. The hypothalamus in the brain recognizes these rising levels and releases GnRH. This hormone prompts the pituitary gland to produce raised levels of LH and FSH. In response to this signaling, the subject may endogenously secrete levels of LH between 20 and 60 IU/L (e.g., between 20 and 25 IU/L, between 25 and 30 IU/L, between 30 and 35 IU/L/between 35 and 40 IU/L, between 40 and 45 IU/L, between 45 and 50 IU/L, between 50 and 55 IU/L, or between 55 and 60 IU/L; e.g., 20 IU/L, 21 IU/L, 22 IU/L, 23 IU/L, 24 IU/L, 25 IU/L, 26 IU/L, 27 IU/L, 28 IU/L, 29 IU/L, 30 IU/L, 31 IU/L, 32 IU/L, 33 IU/L, 34 IU/L, 35 IU/L, 36 IU/L, 37 IU/L, 38 IU/L, 39 IU/L, 40 IU/L, 41 IU/L, 42 IU/L, 43 IU/L, 44 IU/L, 45 IU/L, 46 IU/L, 47 IU/L, 48 IU/L, 49 IU/L, 50 IU/L, 51 IU/L, 52 IU/L, 53 IU/L, 54 IU/L, 55 IU/L, 56 IU/L, 57 IU/L, 58 IU/L, 59 IU/L, or 60 IU/L). During ovulation, the egg is released from its follicle, but the ruptured follicle stays on the surface of the ovary. For the next two weeks or so, the follicle transforms into a structure known as the corpus luteum. This structure starts releasing progesterone, along with small amounts of estrogen. This combination of hormones maintains the thickened lining of the uterus, waiting for a fertilized egg to stick (implant). In some embodiments, the OSC implant may primarily secrete levels of progesterone that is consistent with ovulation at a range from 2 to 25 ng/ml of progesterone (e.g., 2 to 5 ng/ml, 5 to 10 ng/ml, 10 to 15 ng/ml, 15 to 20 ng/ml, or 20 to 25 ng/ml; e.g., 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, 21 ng/ml, 22 ng/ml, 23 ng/ml, 24 ng/ml, or 25 ng/ml).

In some embodiments, the secretion of one or more hormones may stop based on chemical signaling transmitted from the hypothalamus through the bloodstream.

In some embodiments, an OSC implant secretes hormones that correlate with a subject's body-mass index (BMI), such that a subject with a higher BMI has an OSC implant that secretes higher levels of hormones compared to a reference subject with a lower BMI. In some embodiments, an OSC implant comprises a greater number of cells compared to a reference subject with a lower BMI.

### C. Subject Selection

The methods of administering an OSC implant described herein may be directed to a subject seeking IVF treatment options due to ovarian decline, among other reasons. In general, a subject is a female with a low oocyte retrieval number or a subject with many immature oocytes or reduced ovarian hormone levels due to reduced response to natural hormone fluctuations in the body. A subject may be between 20 and 45 years old, and a subject is typically 35 years of age or older. A subject may have ovarian decline and/or a reduced ovarian reserve due to advancing age and/or a genetic or medical condition (e.g., primary ovarian insufficiency (POI), polycystic ovarian syndrome (PCOS), premature menopause, endometriosis, uterine fibroids, gynecological cancer, interstitial cystitis, pelvic inflammatory disease (PID), vaginitis, cervical dysplasia, uterine fibroids, pelvic floor prolapse, interstitial cystitis) that leads to a reduced ovarian reserve. A subject may have an ovarian reserve of 20 or fewer oocytes such that a subject has 1 to 5 oocytes, 4 to 10 oocytes, 8 to 16 oocytes, or 15 to 20 oocytes, e.g., the subject has 1 oocyte, 2 oocytes, 3 oocytes, 4 oocytes, 5 oocytes, 6 oocytes, 7 oocytes, 8 oocytes, 9 oocytes, 10 oocytes, 11 oocytes, 12 oocytes, 13 oocytes, 14 oocytes, 15 oocytes, 16 oocytes, 17 oocytes, 18 oocytes, 19 oocytes, or 20 oocytes. A subject may have anti-Mullerian hormone (AMH) levels that are consistent with reduced ovarian reserve. A subject may have their AMH levels measured by a blood test and other methods known in the art. A subject may have AMH levels between 1 and 6 ng/ml (e.g., 1-2 ng/ml, 2-4 ng/ml, or 4-6 ng/ml; e.g., 1 ng/ml, 2 ng/ml, 3 ng/ml, 4 ng/ml, 5 ng/ml, or 6 ng/ml). A subject may have measured estradiol levels between 20 and 50 pg/ml (e.g., 20-30 pg/ml, 25-35 pg/ml, 30-40 pg/ml, 35-45 pg/ml, or 40-50 pg/ml; e.g., 20 pg/ml, 21 pg/ml, 22 pg/ml, 23 pg/ml, 24 pg/ml, 25 pg/ml, 30 pg/ml, 35 pg/ml, 40 pg/ml, 45 pg/ml, or 50 pg/ml).

A physician or skilled practitioner may evaluate a subject for the methods of administering an OSC implant by taking a biological sample from the subject. A biological sample may include a laboratory specimen held by a biorepository for research. In some embodiments, a biological sample may include bodily fluids including blood, saliva, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, optic fluid, gastric fluid, breast milk, cell culture supernatants, and the like. A biological sample may include a medical diagnosis, user input describing how a user is feeling and/or a symptomatic complaint, information collected from a wearable device pertaining to a user and the like. For example, a biological sample may include information obtained from a visit with a medical professional such as a health history. In yet another non-limiting example, a biological sample may include information such as data collected from a wearable device worn by a user and designed to collect information relating to a user's sleep patterns, exercise patterns, and the like. In an embodiment, a biological sample collected at a particular date and/or time of a user's menstrual cycle. For instance, and without limitation, a biological sample may be collected on the second day of a user's menstrual cycle to evaluate one or more hormone levels. The biological sample may be utilized to determine markers of a subject's ovarian reserve that may be measured by a subject's AMH levels and/or other hormone levels or other indications. AMH levels of 1 ng/ml or less may be used to indicate a low ovarian reserve. A subject with a low ovarian reserve may have measured AMH levels of 1.0 ng/ml, 0.9 ng/ml, 0.8 ng/ml, 0.7 ng/ml, 0.6 ng/ml, 0.5 ng/ml, 0.4 ng/ml, 0.3 ng/ml, 0.2 ng/ml, or 0.1 ng/ml. Other biological samples that may be utilized to determine one or more markers of a subject's overall health include without limitation menstrual cycle progression, and/or monitor circulating hormone levels such as estradiol (E2), luteinizing hormone (LH), follicle-stimulating hormone (FSH), progesterone (P4), estrone (E1), estriol (E3), testosterone, androgens, dehydroepiandrosterone (DHEA), triiodothyronine (T3), tetraiodothyronine (T4), calcitonin, melatonin, insulin, cortisol, human growth hormone (HGH), adrenaline levels, and other hormones.

Other biological sample data taken from a subject may include an oocyte. As used in this disclosure, “biological sample data” is data that provides a characterization of the biological, genetic, biochemical and/or physiological properties, compositions, or activities of biological samples. In some embodiments, an oocyte may be an immature oocyte. As is described in the definitions recited above, an “immature oocyte” as used in this disclosure is an immature reproductive cell originating in the ovaries. In some embodiments, an immature oocyte may be an oocyte including GV and/or MI oocytes. In some embodiments, an immature oocyte may be a plurality of oocytes. An immature oocyte may be immature cumulus-oocyte complexes (COCs) taken from the subject. As used in this disclosure, a “cumulus-oocyte complex” is an oocyte surrounded by specialized granulosa cells. As used in this disclosure, a “specialized granulosa cell” is a cumulus cell surrounding the oocyte to ensure healthy oocyte and embryo development.

In some embodiments of the method, the biological sample may be extracted from the user through an extraction device. An “extraction device” is a device and/or tool capable of obtaining, recording and/or ascertaining a measurement associated with a sample. The extraction device may include a needle, syringe, vial, lancet, Evacuated Collection Tubes (ECT), tourniquet, vacuum extraction tube systems, any combination thereof and the like. For example, the extraction device may comprise a butterfly needle set. Data from a biological sample may include measurements, for example, of serum calcium, phosphate, electrolytes, blood urea nitrogen and creatinine, uric acid, and the like.

In an embodiment of the method, biological sample information of a subject may be obtained from an ultrasound. An “ultrasound,” as used in this disclosure, is any procedure that utilizes sound waves to generate one or more images of a user's body. For example, an ultrasound may be utilized to obtain an image of a subject's reproductive organs and/or tissues. In an embodiment, an ultrasound may be performed at a particular time of a subject's menstrual cycle. For example, a subject may receive an ultrasound on day 2 of her cycle and this may be utilized to determine follicle size and/or follicle count.

Selection of a stimulation protocol and/or adjustment to a stimulation protocol may be made utilizing this information. For example, a subject with an ultrasound that shows PCOS may have a dose adjustment made to one or more medications received and/or utilized during a stimulation protocol. In addition, the length of her stimulation protocol may be modified based on her PCOS diagnosis. In an embodiment, an ultrasound may be repeated one or more times throughout a subject's stimulation protocol, and information obtained may be utilized to adjust her stimulation protocol in real time.

Prior to receiving an OSC implant, a subject's serum may be evaluated for levels of hormones or other relevant compounds. A subject may have low serum levels of estradiol with levels of estradiol lower than 20 pg/ml (e.g., lower than 20 pg/ml, lower than 19 pg/ml, lower than 18 pg/ml, lower than 17 pg/ml, lower than 16 pg/ml, lower than 15 pg/ml, lower than 14 pg/ml, lower than 13 pg/ml, lower than 12 pg/ml, lower than 11 pg/ml, lower than 10 pg/ml, or lower) prior to receiving an OSC implant. A subject who has had an ovariectomy, or the removal or partial removal of their ovaries may have estradiol levels lower than 10 pg/ml (e.g., lower than 10 pg/ml, lower than 9 pg/ml, lower than 8 pg/ml, lower than 7 pg/ml, lower than 6 pg/ml, lower than 5 pg/ml, lower than 4 pg/ml, lower than 3 pg/ml, lower than 2 pg/ml, lower than 1 pg/ml, or even lower) prior to receiving an OSC implant. A subject may have low serum levels of progesterone prior to receiving an OSC implant. In some embodiments a subject may have serum levels of progesterone lower than 0.5 ng/ml (e.g., lower than 0.5 ng/ml, lower than 0.4 ng/ml, lower than 0.3 ng/ml, lower than 0.2 ng/ml, lower than 0.1 ng/ml, or lower) prior to receiving an OSC implant. Further, a subject may have serum levels of LH from about 1.0 mlU/ml to about 2.5 mlU/ml (e.g., from about 1.0 mlU/ml to about 1.5 mlU/ml, from about 1.5 mlU/ml to about 2.0 mlU/ml, or from about 2.0 mlU/ml to about 2.5 mlU/ml; e.g., about 1.0 mlU/ml, about 1.25 mlU/ml, about 1.5 mlU/ml, about 1.75 mlU/ml, about 2 mlU/ml, about 2.25 mlU/ml, or about 2.5 mlU/ml) prior to receiving an OSC implant. A subject may have serum levels of FSH from about 11 mlU/ml to about 14 mlU/ml (e.g., from about 11 mlU/ml to about 12 mlU/ml, from about 12 mlU/ml to about 13 mlU/ml, or from about 13 mlU/ml to about 14 mlU/ml; e.g., about 11 mlU/ml, about 12 mlU/ml, about 13 mlU/ml, or about 14 mlU/ml) prior to receiving an OSC implant.

### D. Sites of Administration

An OSC implant may be applied by a healthcare provider (e.g., a physician, an OB/GYN, a nurse practitioner, or other skilled practitioner) using multiple possible routes of administration. A healthcare provider may use a subject's preference or biological or medical data to determine the best site for OSC implantation.

A physician or skilled practitioner may determine the administration of an OSC implant directed to a subject using the described biological parameters. Such biological parameters include hormone levels (e.g., baseline hormone levels and/or hormone levels due to use of contraceptives), subject anatomy (e.g., follicle size, follicle count, ovarian morphology, and/or uterine morphology), subject body-mass index (BMI), among other biological parameters known to a skilled practitioner. A skilled practitioner may administer a stimulation protocol with any one or a combination of triggering agents, or compositions directed to stimulate follicular maturation and oocyte release, described herein.

Hormone levels or concentrations of other relevant compounds of the biological sample may include estradiol (E2), luteinizing hormone (LH), follicle-stimulating hormone (FSH), progesterone (P4), estrone (E1), estriol (E3), testosterone, androgens, dehydroepiandrosterone (DHEA), triiodothyronine (T3), tetraiodothyronine (T4), calcitonin, melatonin, insulin, cortisol, human growth hormone (HGH), adrenaline levels and the like. In some embodiments, the measurement of hormone levels may be based on blood analysis of the biological sample. For example, blood analysis may include plasma hormone analysis techniques. In some embodiments, measurement of hormone levels may be based on saliva hormone testing techniques. Measurement of hormone levels may be based on other forms of analysis such as hair, urine, and any other form of biological samples described throughout this disclosure. A subject may have a baseline serum level of estradiol from about 30 pg/ml to about 60 pg/ml (e.g., from about 30 pg/ml to about 45 pg/ml, from about 40 pg/ml to about 55 pg/ml, or from about 45 pg/ml to about 60 pg/ml; e.g., about 30 pg/ml, about 35 pg/ml, about 40 pg/ml, about 45 pg/ml, about 50 pg/ml, about 55 pg/ml, or about 60 pg/ml) prior to the follicular triggering period. A subject may have a baseline serum level of progesterone from about 0.5 ng/ml to about 2.5 ng/ml (e.g., from about 0.5 ng/ml to about 1.0 ng/ml, from about 1.0 ng/ml to about 1.5 ng/ml, from about 1.5 ng/ml to about 2.0 ng/ml, or from about 2.0 ng/ml to about 2.5 ng/ml; e.g., about 1.0 ng/ml, about 1.5 ng/ml, about 2.0 ng/ml, or about 2.5 ng/ml) prior to the follicular triggering period.

Additionally, a subject's contraception (e.g., hormonal contraception) usage may affect administration of an OSC implant. Consideration for contraception may aid in determining the administration of an OSC implant.

Insertion of an OSC implant may follow health and safety protocols known in the art.

Considerations related to health and safety may include one or more extractions of a biological sample for a measurement of a hormone described herein. Other considerations may include sterilizing the administration site, numbing the administration site, and/or administering local or systemic analgesics to the subject prior to or upon implantation.

In some embodiments, an OSC implant may be inserted subdermally in the upper arm or the abdomen of a subject. In some embodiments, the OSC implant may be inserted at the inner side of the non-dominant upper arm about 8-10 cm or 3-4 inches (e.g., about 8 cm, about 8.5 cm, about 9 cm, about 9.5 cm, about 10 cm) above the medial epicondyle of the humerus to reduce the risk of neural or vascular injury. In some embodiments, the OSC implant is applied to the subject with a needle (e.g., a hypodermic needle) or other applicator compatible with subdermal insertion.

In some embodiments, an OSC implant may be inserted or administered onto the ovary. In other embodiments, an OSC implant may be inserted or administered onto or adjacent to the fallopian tubes. In some embodiments, an OSC implant is applied as a patch onto the desired tissue (e.g., a microneedle patch, a reservoir patch, a single layer patch, a multi-layer patch, among other examples). In some embodiments, an OSC implant is applied to the desired tissue via direct injection with a tissue-specific applicator (e.g., a vaginal cylinder applicator or an ovoid applicator).

In some embodiments, an OSC implant may be inserted as an intrauterine device (IUD), which is a small, often ‘T’-shaped contraceptive device that is implanted into the uterus using, e.g., methods known in the art. In some embodiments, the IUD can be hormonal or non-hormonal are long-acting, reversible. In some embodiment, the OSC implant may be porous implant (e.g., comprised of porous material such as titanium), which is characterized by the presence of regular or stochastic voids in the metallic or ceramic matrix depending on their manufacture.

### E. Implant Half-Life

An OSC implant is designed to be surgically inserted into a subject's body as described in the above section and is, as such, designed to be long lasting as compared to presently available treatment options for those seeking ART or HRT, which involve frequent medical appointments and continuous injections of hormones. An OSC implant, in contrast, offers many practical benefits and is therefore a potentially more attractive treatment option with fewer doctor appointments, fewer prescription refills, and no daily or weekly injections of hormones, among other potential benefits to the subject.

In some embodiments, an OSC implant retains its hormone-secreting function following implantation into a subject for a time period that is at least 200 days (e.g., at least 200 days, at least 210 days, at least 220 days, at least 230 days, at least 240 days, at least 250 days, at least 260 days, at least 270 days, at least 280 days, at least 290 days, at least 300 days, or longer). In some embodiments, an OSC implant retains its hormone-secreting function following implantation into a subject for a time period that is at least 6 months (e.g., at least 6 months, at least 7 months, at least 8 months, at least 9 months, at least 10 months, at least 11 months, at least 12 months, at least 13 months, at least 14 months, at least 15 months, or longer). In some embodiments an OSC implant retains its hormone-secreting function following implantation into a subject for a time period between 12 and 24 months (e.g., between 12 and 15 months, between 15 and 18 months, between 18 and 21 months, or between 21 and 24 months; e.g., 12 months, 13 months, 14 months, 15 months, 16 months, 17 months, 18 months, 19 months, 20 months, 21 months, 22 months, 23 months, or 24 months). such as 3 months and/or 3 years. In some embodiments, the OSC implant is removed and/or by a skilled medical practitioner after a certain period of time.

### F. Implant Kits

In some embodiments, the compositions and methods described herein also features one or more kits. A kit may contain the compositions described herein or other devices related to implantation.

Such kits may be used for therapeutic or research purposes.

### EXAMPLES

### Example 1. Exemplary Method of an Implant for Ovarian Decline

At a high level, aspects of the present disclosure are directed to apparatuses (e.g., implants) and methods for treating, ameliorating, and/or altogether reversing ovarian decline (e.g., by using the apparatuses (e.g., implants)).

Aspects of the present disclosure can be used to reverse ovarian decline in a person by providing personalized cyclic dosing of hormones into a user. Aspects of the present disclosure can also be used as an ectopic ovarian hub to secrete replacement hormones into a user.

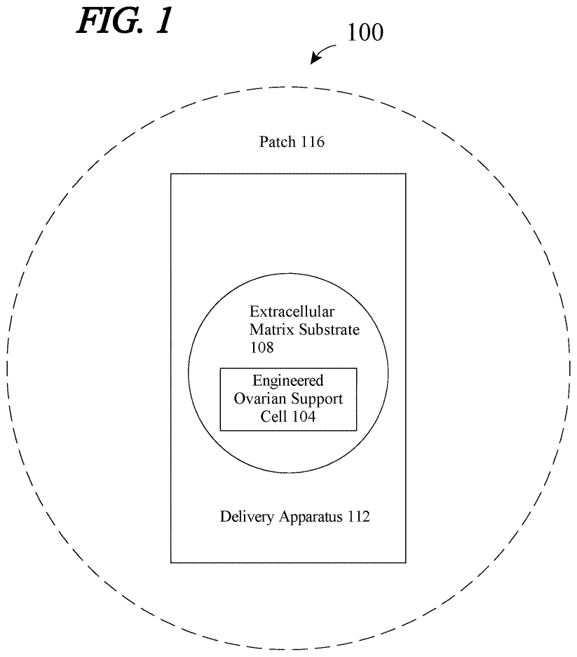

Referring now to the drawings, FIG. 1 illustrates an exemplary method of an implant 100 for ovarian decline. The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve embodiments according to this disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

Still referring to FIG. 1, an “implant,” as used in this disclosure, is a device applied to and/or inserted into a living being. A “living being,” as used in this disclosure, is any organism or life form that is alive. A living being may include a human being, an animal, a plant, a fungus, and the like. An implant may replace a missing biological structure, support a damaged biological structure, and/or enhance an existing biological structure of a living being. An implant may deliver a therapeutically effective amount of a substance directly to a target tissue and/or the bloodstream. A “substance,” as used in this disclosure, is a matter of particular or definite chemical constitution. A substance may be a drug product, or biological substance such as engineered ovarian support cell (described further below). For example, a drug product may include hormones such estrogen, progesterone, and/or testosterone. A dug product may include a class of antidepressants, anticonvulsants, anesthetics, anti-addiction agents, antibacterial, antidementia agents, and/or antifungals. For example, a substance may include selective serotonin reuptake inhibitors (SSRIs), and/or gabapentin. A drug product may include an herbal remedy like black cohosh, agnus castus, shatavari, withania somnifera, and Maca. Implant 100 may be a medical device made of a biomedical material such as titanium, silicone, apatite, and the like. Implant 100 may deliver medication, monitor body functions, or provide support to organs and tissues. In an embodiment, implant 100 may be placed permanently in a living being. In an embodiment, implant 100 may be removeable. “Permanently,” as used in this disclosure is the placement of an implant as a bio-structure substitute. For example, permanent dentures are false teeth that are affixed to the bone in the jaw. These implant dentures are not removable like common dentures. Implant 100 may act as a permanent ectopic ovarian hub to replace the function of a user's ovary in hormone production. “Removable,” as used in this disclosure, is the temporary placement of implant to carry out a specific function. For example, contraceptive implants may be placed in a user only for a certain period, such as 5 years, before being removed. Implant 100 may be removed from a user after reversing or stabilizing symptoms or levels of ovarian decline. For example, implant 100 may be removed after an hour, day, week, and/or month.

Still referring to FIG. 1, in an embodiment, implant 100 may be a contraceptive implant. A “contraceptive implant,” as used in this disclosure, is an implantable medical device used for the purpose of birth control. “Birth control,” as used in this disclosure, is any method, medicine, or device used to prevent pregnancy. Other uses of a contraceptive implant may include, regulating menstrual cycles, preventing ovarian cysts, relieving premenstrual syndrome symptoms, relieving polycystic ovarian syndrome (PCOS) symptoms, relieving dysmenorrhea symptoms, and lowering migraines. Implant 100 may depend on the timed release of hormones to hinder ovulation or sperm development, the ability of copper to act as a natural spermicide within the uterus, or it may work using a non-hormonal, physical blocking mechanism. For example, implant 100 may be an intrauterine device (IUD), which as used in this disclosure, is a small contraceptive device, often ‘T’-shaped, which is implanted into the uterus. IUD can be hormonal or non-hormonal are long-acting, reversible. In an embodiment, implant 100 may be porous implant. A porous implant, as used in this disclosure, is an implant made of porous material such as titanium. Porous implants are characterized by the presence of voids in the metallic or ceramic matrix. Voids can be regular, such as in additively manufactured (AM) lattices, or stochastic, such as in gas-infiltrated production processes.

Still referring to FIG. 1, implant 100 is utilized for treating ovarian decline. “Ovarian decline,” as used in this disclosure, is a decline in ovarian function. A decline in ovarian function may cause a female to lose her normal reproductive potential. A loss of normal reproductive potential may manifest as one or more conditions including but not limited to infertility, primary ovarian insufficiency (POI), poly cystic ovarian syndrome (PCOS), menopause, endometriosis, uterine fibroids, gynecological cancer, interstitial cystitis, pelvic inflammatory disease (PID), vaginitis, cervical dysplasia, uterine fibroids, pelvic floor prolapse, interstitial cystitis, and the like. Ovarian decline may manifest in a variety of uncomfortable or even life threating conditions. In medicine, “comorbidity” is the presence of one or more additional conditions often co-occurring with a primary condition. Comorbidity describes the effect of all other conditions an individual patient might have other than the primary condition of interest and can be physical or psychological. Comorbidities of ovarian decline may include but is not limited to dementia, depression, strokes, cardiovascular disease, arthritis, osteoporosis, anxiety, menopause, hypothyroidism, low libido, and similar conditions. “Dementia,” as used in this disclosure, is a chronic or persistent disorder of the mental processes caused by brain disease or injury and marked by memory disorders, personality changes, and impaired reasoning. If the body does not produce sufficient levels of hormones (i.e., thyroid hormone), the neurotransmitters cannot function properly. Memory span and ability to concentrate will be negatively affected. As the body's estrogen levels decrease, which happens before and during menopause, estrogen can no longer participate in brain function as it normally had. This can cause occasional lapses in brain function, resulting in short-term memory issues. “Depression,” as used in this disclosure, is a mood disorder that causes a persistent feeling of sadness and loss of interest. The reduction, continued deficit, and or changes in levels of estrogens throughout the menstrual cycle are correlated with mood-related distress. A “stroke,” as used in this disclosure, is a biological occurrence where something blocks blood supply to part of the brain or when a blood vessel in the brain bursts. A long period of estrogen deprivation can lead to loss of sensitivity and protective effects in the brain and weaken areas normally resistant to stroke damage.

Still referring to FIG. 1, in an embodiment, implant 100 may contain electronic artificial pacemakers, cochlear implants, and the like. Implant 100 may be bioactive, such as a subcutaneous drug delivery device in the form of implantable pills or drug-eluting stents. Implant 100 may be used in a sensory/neurological based application for disorders affecting the major senses and the brain, as well as other neurological disorders. For example, implant 100 may be used in the treatment of conditions such as cataract, glaucoma, keratoconus, and other visual impairments; otosclerosis and other hearing loss issues, as well as middle ear diseases such as otitis media; and neurological diseases such as epilepsy, Parkinson's disease, and treatment-resistant depression.

Still referring to FIG. 1, in an embodiment, implant 100 may be a transdermal patch. A transdermal patch, as used in this disclosure, is a medicated adhesive patch that is placed on the skin to deliver a specific dose of any substance, as described above, through the skin and into the bloodstream. For example, implant 100 may be a single-layer drug-in-Adhesive, the adhesive layer of this system also contains the drug. In this type of patch, the adhesive layer not only serves to adhere the various layers together, along with the entire system to the skin, but is also responsible for the releasing of the drug. The adhesive layer is surrounded by a temporary liner and a backing. It is characterized by the inclusion of the drug directly within the skin-contacting adhesive placed onto the epidermis. Implant 100 may be a Multi-layer Drug-in-Adhesive. The multi-layer drug-in-adhesive patch is similar to the single-layer system; the multi-layer system is different, however, in that it adds another layer of drug-in-adhesive, usually separated by a membrane (but not in all cases). One of the layers is for immediate release of the drug and other layer is for control release of drug from the reservoir. This patch also has a temporary liner-layer and a permanent backing. The drug release from this depends on membrane permeability and diffusion of drug molecules. Implant 100 may be a reservoir type patch. Unlike the single-layer and multi-layer drug-in-adhesive systems, the reservoir transdermal system has a separate drug layer. The drug layer is a liquid compartment containing a drug solution or suspension separated by the adhesive layer. The drug reservoir is totally encapsulated in a shallow compartment molded from a drug-impermeable metallic plastic laminate, with a rate-controlling membrane made of a polymer like vinyl acetate on one surface. This patch is also backed by the backing layer. In this type of system, the rate of release is zero order. Implant 100 may be a matrix type patch. The matrix system has a drug layer of a semisolid matrix containing a drug solution or suspension. The adhesive layer in this patch surrounds the drug layer, partially overlaying it. Also known as a monolithic device. Implant 100 may be a vapor patch. In a vapor patch, the adhesive layer not only serves to adhere the various layers together but also to release vapor.

Still referring to FIG. 1, implant 100 may be a microneedle patch (MNP). A microneedle patch, as used in this disclosure, is a transdermal patch including microneedles as the drug delivery system. For example, implant 100 may be a dissolvable MNP, solid non-soluble MNP, and/or a hollow MNP. In some embodiments, implant 100 may be topical: powder, lotion liniment, solution, emulsion, suspension, ointment, cream, liquid, pastes, gel, and/or suppository. In some embodiments, implant 100 may be in the form of digestible tablets. In some embodiments, implant 100 may be a polymeric drug delivery system, which as used in the disclosure, is a formulation or a device that enables the introduction of a therapeutic substance into the body. For example, implant 100 may a polymeric vaginal ring.

Still referring to FIG. 1, “cardiovascular diseases (CVDs),” as used in this disclosure, are a group of disorders of the heart and blood vessels. CVDs include the following ailments: coronary heart disease—a disease of the blood vessels supplying the heart muscle; cerebrovascular disease-a disease of the blood vessels supplying the brain; peripheral arterial disease—a disease of blood vessels supplying the arms and legs; rheumatic heart disease-damage to the heart muscle and heart valves from rheumatic fever, caused by streptococcal bacteria; congenital heart disease-birth defects that affect the normal development and functioning of the heart caused by malformations of the heart structure from birth; and deep vein thrombosis and pulmonary embolism-blood clots in the leg veins, which can dislodge and move to the heart and lungs. Lower levels of estrogen can affect the muscles lining the arteries and can increase the buildup of cholesterol in the arteries. These factors increase a person's risk of atherosclerosis (hardening of the arteries). “Hypothyroidism,” as used in this disclosure, is a common condition where the thyroid doesn't create and release enough thyroid hormone into one's bloodstream. This makes one's metabolism slow down. Also called underactive thyroid, hypothyroidism can make one feel tired, gain weight and be unable to tolerate cold temperatures. “Osteoporosis,” as used in this disclosure, is a disease that thins and weakens the bones. Bones become fragile and fracture (break) easily, especially the bones in the hip, spine, and wrist. The hormone estrogen helps keep bones strong. “Anxiety,” as used in this disclosure, is a mental health disorder characterized by feelings of worry, anxiety, or fear that are strong enough to interfere with one's daily activities. The reduction, continued deficit, and or changes in levels of estrogens throughout the menstrual cycle are correlated with mood-related distress. “Menopause,” as used in this disclosure, is the ceasing of menstruation. Common symptoms include hot flashes and vaginal dryness. There may also be sleep disturbances. The combination of these symptoms can cause anxiety or depression. Estrogen in combination with progesterone and testosterone, promotes growth and maintenance of reproductive organs and keeps the vagina moisturized, elastic (stretchy), and well supplied with blood. When levels of estrogen decline, menopause may occur.

Still referring to FIG. 1, to combat ovarian decline, implant 100 includes at least an engineered ovarian support cell. An “engineered ovarian support cell,” as used in this disclosure, is a reproductive cell originating in an ovary engineered in vitro. “Engineering,” as used in this disclosure, is a process that alters the genetic makeup of an organism. Engineering may include altering the DNA makeup such as for example by changing a single base pair, deleting a region of DNA, adding a new segment of DNA, manipulating DNA, modifying DNA, recombining DNA and/or a nucleic acid, and the like. Engineering may include the design and construction of new biological entities such as with the use of laboratory technologies such as enzymes, genetic circuits, and cells or the redesign of existing biological systems. Engineering may include differentiating an engineered ovarian support cell to express one or more transcription factors. A “transcription factor,” as used in this disclosure is any protein that controls a rate of transcription. For example, a transcription factor may be selected from NR5A1 and a RUNX family protein. For instance, an engineered ovarian support cell may include an engineered granulosa cell configured to express and/or overexpress RUNX 1. An engineered ovarian support cell may express a particular protein and/or transcription factor if a level of the protein is detectable such as for example using a known protein assay. An engineered ovarian support cell may overexpress a particular protein and/or transcription factor if a particular protein and/or transcription factor level is detectable at a higher reference range. For example, an engineered ovarian support cell may overexpress a particular protein if the protein is detectable at a level that is 5% higher than the level of the protein expressed from an endogenous naturally occurring polynucleotide encoding the protein. Engineering may include engineering one or more polynucleotides of an engineered ovarian support cell. An “engineered polynucleotide,” as used in this disclosure, is a nucleic acid that does not occur in nature. An engineered polynucleotide may include a recombinant nucleic acid. A “recombinant nucleic acid,” as used in this disclosure, is a molecule that is constructed by joining nucleic acids from two different organisms. For example, a recombinant nucleic acid may be created from a human and a mouse. An engineered polynucleotide may include a synthetic nucleic acid. A “synthetic nucleic acid,” as used in this disclosure, is a molecule that is amplified and/or chemically synthesized. For example, a synthetic nucleic acid may include a chemically modified and/or otherwise modified nucleic acid that can bind to one or more naturally occurring molecules. An engineered polynucleotide may include DNA (genomic DNA, cDNA, and/or any combination thereof), RNA, and/or a hybrid molecule. An engineered polynucleotide may include complementary DNA which may be synthesized from a single stranded RNA (messenger RNA (mRNA) or microRNA (miRNA)) such as for example using a catalyst such as but not limited to reverse transcriptase. In an embodiment, an engineered polynucleotide may include a promoter operably linked to an open reading frame. A “promoter,” as used in this disclosure, is a nucleotide sequence to which RNA polymerase binds to initiate transcription. A promoter may include an inducible promoter. An inducible promoter may be regulated in vitro by a stimulus such as a chemical agent, temperature, or light. This may allow for temporal and/or spatial control of gene expression. For example, an inducible promoter may include but is not limited to an alcohol regulated promoter, a tetracycline operator sequence, a steroid regulated promoter, a human estrogen receptor, and the like. In an embodiment, engineering may include altering of the cell's ability to express, overexpress and/or secrete a hormone including but not limited to hormones such as estrogen, progesterone, testosterone, DHEA and the like. An engineered ovarian support cell may include but is not limited to an engineered granulosa cell, an engineered lutein cell, and/or an engineered theca cell as described below in more detail. In an embodiment, an engineered ovarian support cell may be sourced from an oocyte, granulosa cell, cumulus oocyte complex, and similar cells originating in the ovary. An “oocyte,” as used in this disclosure, is a female gametocyte or germ cell involved in reproduction. In an embodiment, an engineered ovarian support cell may include an engineered granulosa cell. A “granulosa cell” is an estrogen-secreting cell of the epithelial lining of a graafian follicle and/or or its follicular precursor. Engineering may include any engineering process as described herein. For instance, and without limitation, a granulosa cell may be engineered to overexpress quantities of estradiol. “Estrogen” as used in this disclosure is a steroid hormone that promotes the development and/or maintenance of female sex characteristics. Estrogen may include but is not limited to estrone (E1), estradiol (E2), estriol (E3), estetrol (E4) and/or any combination thereof. A “cumulus oocyte complex,” as used in this disclosure, is an oocyte containing one or more surrounding cumulus cells. A COC may contain an immature oocyte. A COC may contain a mature oocyte. A “mature oocyte” as used in this disclosure, is one or more mature reproductive cells originating in the ovaries. In some embodiments, engineered ovarian support cell 104 may include but is not limited to an engineered cell and/or any combination thereof including oogonia cells, oogonia-like pluripotent stem cells, polynucleotides, primordial germ cells, and primordial germ cell-like pluripotent stem cells. For example, engineered ovarian support cell 104 may include a pluripotent stem cell (PSC) incorporating: an engineered polynucleotide including an open reading frame encoding a protein selected from Zinc Finger Protein 281 (ZNF281), LIM Homeobox 8 (LHX8), and Spermatogenesis and Oogenesis Specific Basic Helix-Loop-Helix 1 (SOHLH1). In yet another non-limiting example, engineered ovarian support cell 104 may include a pluripotent stem cell (PSC) incorporating an engineered polynucleotide including an open reading frame encoding a protein selected from Distal-Less Homeobox 5 (DLX5), Hematopoietically-expressed homeobox protein (HHEX), and Folliculogenesis Specific BHLH Transcription Factor (FIGLA). In yet another non-limiting example, engineered ovarian support cell 104 may include a pluripotent stem cell (PSC) incorporating an engineered polynucleotide including an open reading frame encoding a protein selected from nuclear receptor subfamily 5 group A member 1 (NR5A1) and a Runt-related transcription factor (RUNX) family protein.

Still referring to FIG. 1, in an embodiment, an ovarian cell may be engineered using Clustered Regularly Interspaced Short Palindromic Repeats (CRISPR) technology. “CRISPR” is programmable technology that targets specific stretches of genetic code to edit DNA at precise locations. CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platforms and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of IN vitro Transcription (SPRINT). SPRINT can be used to detect a variety of substances, such as metabolites in patient samples or contaminants in environmental samples, with high throughput or with portable point-of-care devices.

Still referring to FIG. 1, in some embodiments, engineered ovarian support cell 104 may be derived from a pluripotent stem cell. “Pluripotent stem cells,” as used in this disclosure, are cells that are able to self-renew by dividing and developing into the three primary groups of cells that make up a human body, including ectoderm, giving rise to the skin and nervous system; endoderm, forming the gastrointestinal and respiratory tracts, endocrine glands, liver, and pancreas; and mesoderm, forming bone, cartilage, most of the circulatory system, muscles, connective tissue, and more. Pluripotent stem cells may be able to make cells from all three of these basic body layers, so they can potentially produce any cell or tissue the body needs to repair itself. Pluripotent stem cells may include induced pluripotent stem cells (iPSCs), which are derived from skin or blood cells that have been reprogrammed back into an embryonic-like pluripotent state that may enable the development of an unlimited source of any type of human cell needed for therapeutic purposes. For example, iPSC can be prodded into becoming beta islet cells to treat diabetes, blood cells to create new blood free of cancer cells for a leukemia patient, or neurons to treat neurological disorders. Induced pluripotent cells may be derived from embryos, embryonic stem cells made by somatic cell nuclear transfer (ntESCs) and/or an embryonic stem cell from an unfertilized egg. In an embodiment, a pluripotent cell may include a human pluripotent cell. In an embodiment, a pluripotent cell may include an embryonic stem cell, such as a human embryonic stem cell. An “embryonic stem cell,” as used in this disclosure, is a pluripotent stem cell made using embryos or eggs. An embryonic stem cell may include but is not limited to a true embryonic stem cell, a nuclear transfer embryonic stem cell, and/or a parthenogenetic embryonic stem cell. In an embodiment, a pluripotent stem cell may include an induced pluripotent stem cell such as a human induced pluripotent stem cell. A human induced pluripotent stem cell may be derived from skin or blood cells that may be engineered back into an embryonic-like pluripotent state that enables the development of an unlimited source of any type of human cell.

Still referring to FIG. 1, in some embodiments, engineered ovarian support cell 104 may include an engineered theca cell. A “theca cell,” as used in this disclosure, is one or more endocrine cells associated with ovarian follicles that produce androgens. Engineering may include any engineering process as described herein. An engineered theca cell may be engineered to produce an androgen substrate required for ovarian estrogen biosynthesis, provide signal transduction between granulosa cells and oocytes, provide nutrients, provide structure and support for a follicle as it matures and the like.

Theca cells respond to luteinizing hormone (LH) and produce androgens, as well as progesterone, in the pre-ovulatory large follicles. An “androgen,” as used in this is any natural or synthetic steroid hormone that regulates the development and maintenance of male characteristics in vertebrates by binding to androgen receptors. For example, an androgen may include testosterone, dehydroepiandrosterone (DHEA), androstenedione, dehydroepiandrosterone sulfate (DHEA-S), and/or dihydrotestosterone.

Androgens are synthesized in the testes, the ovaries, and the adrenal glands. Androgens in females may serve one or more functions including aiding libido and sexual arousal. Androgens have potential roles in relaxation of the myometrium via non-genomic, androgen receptor-independent pathways, preventing premature uterine contractions in pregnancy. Also, androgens are the precursors to estrogens in both men and women. In addition to their role as natural hormones, androgens are used as medications in hormone replacement therapy. Low androgen levels relating to ovarian decline produce effects such as low libido (interest in or desire for sex), fatigue, decreased sense of well-being and increased susceptibility to bone loss, osteoporosis and fractures. “Progesterone,” as used in this disclosure, is a steroid hormone belonging to a class of hormones called progestogens. Progesterone prepares the endometrium for the potential of pregnancy after ovulation. It triggers the lining to thicken to accept a fertilized egg. It also prohibits the muscle contractions in the uterus that would cause the body to reject an egg. Low levels of progesterone, in relation to ovarian decline, may cause abnormal menstrual cycles or infertility because the progesterone does not trigger the proper environment for a conceived egg to grow. An implant of the disclosure may be used to supplement hormone levels (e.g., progesterone levels) and, thus, may be used to regulate a menstrual cycle or improve fertility.

Still referring to FIG. 1, in some embodiments, the engineered cell may be a lutein cell. A “lutein cell,” as used in this disclosure, is a cell of the corpus luteum. Engineering may include any engineering process as described herein. An engineered lutein cell may contain lipid droplets and may be derived from granulosa cells and/or the cells of the theca interna. An engineered lutein cell may be engineered to produce and/or secrete predetermined levels of one or more hormones such as progesterone, estrogen, and/or inhibin. “Estrogen,” as used in this disclosure, is a category of sex hormone responsible for the development and regulation of the female reproductive system and secondary sex characteristics Estrogens are responsible for maturation and maintenance of the vagina and uterus, and are also involved in ovarian function, such as maturation of ovarian follicles. In addition, estrogens play an important role in regulation of gonadotropin secretion. For these reasons, estrogens are required for female fertility. Sudden estrogen withdrawal, fluctuating estrogen, and periods of sustained low estrogen levels correlate with significant mood lowering. Estrogens are involved in libido (sex drive) in both women and men. Low levels of estrogen, in relation to ovarian decline, may additionally cause menstrual periods that are less frequent or that stop, hot flashes (suddenly feeling very warm) and/or night sweats, trouble sleeping, dryness and thinning of the vagina, low sexual desire, mood swings, dry skin, weight gain, mainly in your waist, hips, and thighs, menstrual problems, such as light or heavy bleeding, worsening of premenstrual syndrome, fibrocystic breasts (non-cancerous breast lumps), fibroids (noncancerous tumors) in the uterus, fatigue loss of sex drive, depression, osteoporosis, anxiety, and similar conditions. An implant of the disclosure may be used to supplement hormone levels (e.g., estrogen levels) and, thus, may be used to improve one or more of these conditions associated with low estrogen levels.

Still referring to FIG. 1, there are three major endogenous estrogens that have estrogenic hormonal activity: estrone (E1), estradiol (E2), and estriol (E3). Estrone is a steroid, a weak estrogen, and a minor female sex hormone. Estrone, as well as the other estrogens, are synthesized from cholesterol and secreted mainly from the gonads, though they can also be formed from adrenal androgens in adipose tissue. Relative to estradiol, both estrone and estriol have far weaker activity as estrogens. Estrone can be converted into estradiol and serves mainly as a precursor or metabolic intermediate of estradiol. It is both a precursor and metabolite of estradiol. Estradiol is the most potent and prevalent. It can treat symptoms caused by menopause or removal of the ovaries. It can also treat prostate and breast cancer and prevent osteoporosis. Estradiol is responsible for the development of female secondary sexual characteristics such as the breasts, widening of the hips and a female-associated pattern of fat distribution. It is also important in the development and maintenance of female reproductive tissues such as the mammary glands, uterus and vagina during puberty, adulthood and pregnancy. In the female, estradiol acts as a growth hormone for tissue of the reproductive organs, supporting the lining of the vagina, the cervical glands, the endometrium, and the lining of the fallopian tubes. It enhances growth of the myometrium. Estradiol appears necessary to maintain oocytes in the ovary. During the menstrual cycle, estradiol produced by the growing follicles triggers, via a positive feedback system, the hypothalamic-pituitary events that lead to the luteinizing hormone surge, inducing ovulation. In the luteal phase, estradiol, in conjunction with progesterone, prepares the endometrium for implantation. By supplementing and/or restoring the subject's ability to secrete estradiol and progesterone in a manner that is responsive to naturally secreted HPG axis hormones, the implants of the disclosure may augment the subject's ability to prepare the endometrium for successful embryo implantation.

Still referring to FIG. 1, estriol is a steroid, a weak estrogen, and a minor female sex hormone. Levels of estriol in women who are not pregnant are almost undetectable. However, during pregnancy, estriol is synthesized in very high quantities by the placenta and is the most produced estrogen in the body by far, although circulating levels of estriol are similar to those of other estrogens due to a relatively high rate of metabolism and excretion. “Inhibin,” as used in this disclosure, is a protein secreted that obstructs the synthesis and release of the follicle-stimulating hormone (FSH) in the pituitary gland and reduces the hypothalamic luteinizing hormone (LH)-releasing hormone content. In relation to ovarian decline, aging woman may have increased FSH levels during the follicular phase despite normal estrogen and LH levels and regular menstrual cycles. This FSH rise occurs some years before menopause and accompanies the decline of ovarian follicular reserve and fertility rate. The selective FSH rise of late reproductive years might be a consequence of declining inhibin secretion by a reduced pool of ovarian follicles. Advantageously, the implants of the disclosure may restore the subject's ability to secrete estrogen (e.g., estradiol) on a temporal (e.g., cyclic) basis.

Still referring to FIG. 1, in some embodiments, an engineered ovarian support cell may be a follicle-stimulating hormone (FSH) responsive cell. For example, an iPSC-derived (induced pluripotent stem cell derived) granulosa cell capable of producing all hormones herein disclosed, that is steroidogenic when FSH is present at physiologically relevant levels, and non-steroidogenic when it is not, as described further below. “Steroidogenic,” as used in this disclosure are cells that are involved in steroidogenesis and steroid biosynthesis. They are responsible for the biosynthesis of the steroid hormones, including sex steroids (androgens, estrogens, and progestogens) and corticosteroids (glucocorticoids and mineralocorticoids), as well as neurosteroids, from cholesterol. Steroidogenic cells are most highly expressed in classical steroidogenic tissues, such as the testis, ovary, and adrenal cortex, but are also present in other tissues in the body. The engineered ovarian support cell may produce activins, Anti-Müllerian hormone (AMH), and Epidermal growth factor (EGF). “Activins,” as used in this disclosure, are dimeric proteins composed of βA- and βA-subunits (activin A), βB- and βB-subunits (activin B), or βA- and βB-subunits (activin AB). Activins stimulate FSH synthesis and secretion. “Anti-Müllerian hormone,” as used in this disclosure, is a glycoprotein hormone structurally related to inhibin and activin from the transforming growth factor beta superfamily, whose key roles are in growth differentiation and folliculogenesis. AMH is expressed by granulosa cells of the ovary during the reproductive years and limits the formation of primary follicles by inhibiting excessive follicular recruitment by FSH. AMH expression is greatest in the recruitment stage of folliculogenesis, in the preantral and small antral follicles. This expression diminishes as follicles develop and enter selection stage, upon which FSH expression increases. Some authorities suggest it is a measure of certain aspects of ovarian function, useful in assessing conditions such as polycystic ovary syndrome and premature ovarian failure. “Epidermal growth factor,” as used in this disclosure, is a protein that stimulates cell growth and differentiation by binding to its receptor, epidermal growth factor receptor. EGF may be used to modify synthetic scaffolds for manufacturing of bioengineered grafts by emulsion electrospinning or surface modification methods.

Still referring to FIG. 1, engineered ovarian support cell 104 may, in response to a physiological conversation between a user's brain and the user's ovary, secrete key ovarian hormones and provide personalized hormone delivery in real-time as described further below. A “user,” as used in this disclosure is a living organism such as a human being, plant, animal, and all other organisms composed of cells. For example, a user may be a human patient experiencing ovarian decline. The feedback interaction between engineered ovarian support cell 104 and a patient's hormonal environment may mimic pre-menopausal brain signaling relating to the production and release of hormones in the body. A “hormonal environment,” as used in this disclosure are areas in the human body prone to hormone signaling and interaction. The hormonal environment may be the endocrine system in the body, the brain, blood stream, and the like.

For example, steroidogenic cells form a mural granulosa group responding to FSH, while the mixed population (with more of a cumulus identity) may provide the cell-to-cell mechanical environment and response to estradiol (E2) (such as AMH production and human chorionic gonadotropin (hCG) trigger response).

Still referring to FIG. 1, engineered ovarian support cell 104 may be configured to secrete a hormone in a cyclic dose. For example, lutein cells may secrete progesterone. Granulosa cells may secrete estrogen. Theca cells may secrete androgens such as testosterone. The levels and specific secretions of engineered ovarian support cell 104 fluctuate over time and in response to signals from the brain and environment and are unique to each individual user unlike in continuous dosing methods in hormone replacement therapy. Continuous dosing involves a fixed does of a substance being administered to user, not tailored to the physiological relationship between the brain and the ovary which is not how hormones are naturally delivered in the body. Even with precise dose calculators it may be impossible to calculate the ever-fluctuating hormonal requirements of user. What cyclical pattern dosing does is integrate into chemical conversation, respond to signals from brain and ovaries, and tunes levels of hormones released to each individual woman's system. This real time feedback avoids the limitations and side effects of continuous systemic hormonal administration and is essential to prevent the downstream consequences of ovarian decline. Female reproduction relies on a complex balance of hormones that drive the reproductive cycle (menstrual cycle in humans) and influence fertility. A hormone called GnRH, which stands for gonadotropin-releasing hormone, plays a major role in regulating this balance. GnRH is transmitted from the brain and stimulates the release of other hormones from a nearby gland called the pituitary gland, which, in turn, activates the reproductive organs to produce steroid hormones, such as estrogen.

Still referring to FIG. 1, steroids do many things in the body, including regulating the release of GnRH and pituitary hormones through a process called feedback. A “feedback” mechanism, as used in this disclosure, is a loop in which a product feeds back to control its own production. In the case of negative feedback, steroids maintain the release of GnRH and pituitary hormone within a normal range. Once per reproductive cycle, estrogen will instead positively feed back into the system and activate GnRH, causing pituitary hormone levels to spike, and initiate the release of one or more eggs from the ovary by a process known as ovulation. The neurons that make GnRH do not directly respond to estrogen, but instead receive input from different upstream neurons that contain estrogen receptors. For example, treating menopausal symptoms with estrogen and progestin together is known as estrogen-progestin therapy (EPT) or combined hormone therapy. Although estrogen alone improves the symptoms of menopause, it increases the risk of cancer of the uterus (endometrial cancer). Adding a progestin to the estrogen lowers the risk of endometrial cancer back to normal. Because of this, EPT is given to women who still have a uterus (those who have not had a hysterectomy). EPT can be given 2 ways: continuous EPT means the same dose of estrogen and progestin is taken each day. Women often prefer continuous EPT because it rarely leads to menstrual-like bleeding. Sequential (cyclical) EPT means different amounts of each hormone are taken on specific days. There are different ways to do this. For example, estrogen can be taken by itself for 14 days, then estrogen plus progestin for 11 days, then neither hormone for 3 to 5 days. However, these methods do not allow for real time feedback to administer hormones exactly based on brain signaling. EPT is linked to a higher risk of breast cancer.

The longer EPT is used, the higher the risk. In women who still have a uterus, using systemic estrogen therapy has been shown to increase the risk of endometrial cancer (cancer of the lining of the uterus). Therefore, because implant 200 implements cyclic dosing there is a lower chance of cancer risks and other side effects related to HRT since the natural hormones released from the brain will drive the membrane response hormone secretion in the exact way that the brain-ovarian axis is designed, instead of prolonged administration of fixed hormone levels that may be become detrimental to the user in the long term.

Still referring to FIG. 1, With over 1 billion women experiencing menopause today, it is a huge, but underserved market. Estimates suggest that women in menopause spend an average of $2,000 a year on prescriptions, doctor visits, treatments, devices, and products to address symptoms like night sweats, loss of libido, fatigue and difficulty concentrating. Cyclic dosing may be financially advantageous to a user since the dose is personalized and wouldn't require multiple applications, prescriptions, and trials of the average fixed dose hormone products.

Still referring to FIG. 1, implant 100 includes an extracellular matrix substrate 108. In some embodiments, engineered ovarian support cell 104 or a plurality of engineered ovarian support cells 104 may be embedded within extracellular matrix substrate 108, as described further below. “Embed,” as used in this disclosure, is the process of fixing an object in a surrounding mass. For example, embedding may include a cell binding to a collagen receptor in an extracellular matrix. As used in this disclosure, an “extracellular matrix (ECM),” is a non-cellular component that provides physical scaffolding for the cellular constituents and initiates crucial biochemical/biomechanical cues. An “extracellular matrix substrate,” as used in this disclosure, is a network containing one or more extracellular macromolecules and/or minerals that functions to provide structural and/or biochemical support. Extracellular matrix substrate 108 may bind and/or embed one or more ingredients contained within implant 100. Extracellular matrix substrate 108 may include macromolecules and minerals, such as collagen, Epidermal growth factors (EGFs) enzymes, glycoproteins and hydroxyapatite that provide structural and biochemical support to surrounding cells. In some embodiments, an extracellular matrix may include a collagen matrix. An ECM collagen matrix may include a combination of 20 or more different types of collagens. Collagen may include one or more combinations of Types 1-20. Combinations may be based on weight %, quantity of each type used, and the like. For example, collagen I, II, III, V and XI which are fibrillar collagen commonly found in connective tissue, may be used in the ECM, individually or combined. In some embodiments, extracellular matrix substrate 108 may include an elastin. Elastin is hydrophobic protein of 750 amino acids, rich in proline and glycine. Tropoelastin, a soluble precursor secreted into the extracellular space assembles into insoluble elastic fibers and sheets. Elastic fibers provide required resilience so that tissues can recoil after transient stretch. In some embodiments, extracellular matrix substrate 108 may include a fibronectin. Fibronectin is a large glycoprotein (220 kDa) composed of two polypeptide chains (dimer) joined by disulfide bonds at one end. Each polypeptide is further folded into functionally and structurally distinct domains which bind to various components of ECM (glycosaminoglycans, proteoglycans, and collagen) and cell surface proteins. Fibronectin is secreted by wide variety of connective tissue cells, including fibroblasts, chondrocytes, schwann cells, macrophages, intestinal epithelial cells and hepatocytes. Fibronectin is a multifunctional protein involved in cell adhesion and spreading. It also regulates cellular morphology, cell migration, cytoskeletal organization, hemostasis and wound repair.

Still referring to FIG. 1, extracellular matrix substrate 108 may include a laminin. Laminin is major component of basal lamina. It is composed of three long polypeptide chains (designated α, β, and γ) held together by disulfide bonds and arranged in asymmetric cross shape. Laminin acts as glue, which holds cells and the ECM together. It has active domains for collagen binding, cell adhesion, heparin binding and neurite outgrowth fragment. Laminins modulate cell growth, motility and signaling pathways. In some embodiments, extracellular matrix substrate 108 may include vitronectin. Vitronectin is glycoprotein of 459 amino acids, found in ECM and blood. It circulates in blood either in the form of single chain moiety of 75 kDa or as two chain moieties of 65 kDa and 10 kDa. Vitronectin interacts with polysaccharides (Glycosaminoglycans) and proteoglycans, acting as cell adhesion molecule. Although vitronectin and fibronectin have similar functions and have an Arg-Gly-Asp cell recognition sequence, they are structurally and immunologically distinct. Vitronectin acts as inhibitor of cytolytic complement pathway and have physiological role in coagulation pathway. In addition, it promotes cell migration, proliferation, differentiation and spreading of endothelial and neoplastic cells. Other embodiments of extracellular matrix substrate 108 may include proteoglycans, heparan sulfate, chondroitin sulfate, keratan sulfate, hyaluronic acid, elastin, fibronectin, dermatan sulfate, collagen, extracellular vesicles, nanoparticles, microparticles, proteins, cell adhesion proteins, proteoglycans, carbohydrate polymers, non-proteoglycan polysaccharides, and other forms of substrates one skilled in the art may understand as applicable.

Still referring to FIG. 1, the ECM may be a collagen polymer matrix is about 100-500 micron particles. A “collagen polymer matrix,” as used in this disclosure, is a matrix composed of collagen-based macromolecules. A collagen polymer matrix may include collagen types 1-20 as described above individually or in combination. For example, the collagen polymer matrix may be a collagen type III biopolymer matrix derived from fetal, neo-natal and/or post-natal animal tissue. As used in this disclosure, “micron particles,” (microparticles) are spherical particles between 0.1 and 100 μm in size. containing a core substance enclosed by one or more membranes or shells. Microparticles may be further classified as microspheres and microcapsules based on their internal structure. Microspheres are generally formed by a homogeneous matrix in which it is not possible to separate a core and a membrane, while the drug is dispersed in the polymer matrix either as small clusters or molecularly. Microcapsules are formulations constituted by a central liquid, solid, or semisolid core containing the drug, alone, or in combination with excipients, surrounded by a membrane or a continuous polymer coating. Commercially available microparticles are available in a wide variety of materials, including ceramics, glass, polymers, and metals. Microparticles offer various significant advantages as drug delivery systems, including: (i) an effective protection of the encapsulated active agent against (e.g. enzymatic) degradation, (ii) the possibility to accurately control the release rate of the incorporated drug over periods of hours to months, (iii) an easy administration (compared to alternative parenteral controlled release dosage forms, such as macro-sized implants), and (iv) Desired, pre-programmed drug release profiles can be provided which match the therapeutic needs of the patient. In some embodiments, the ECM may contain Poly (lactic acid) (PLA), and/or poly (lactic-co-glycolic acid) (PLGA), microparticles. In some embodiments, the ECM may be a collagen polymer matrix about 500-800 micron cubes. As used in this disclosure, a “micron cube,” is a derived metric SI (System International) measurement unit of volume with sides equal to one micron (1 μm). Additionally, or alternatively, the ECM may be perforated.

Still referring to FIG. 1, implant 100 includes a delivery apparatus. A “delivery apparatus,” as used in this disclosure is a device utilized to deliver a substance to a living being. A “substance” as used in this disclosure includes any substance as described above in more detail. A substance may include an engineered ovarian support cell. A substance may include a drug product. A substance may include a vitamin, supplement, hormone, and the like. A delivery apparatus may be biocompatible with a living being. “Biocompatible,” as used in this disclosure, is the ability of implant 100 to be compatible with a living being while implant 100 is in contact with the living being. Contact may include for example when implant 100 is administered and/or inserted into a living being. For example, implant 100 may be subdermally inserted into a human being's arm where it may be biocompatible with the human being. In an embodiment, implant 100 may be biocompatible with a living being for a certain period of time such as 3 months and/or 3 years. After the expiration of a certain period of time implant 100 may have to be removed. In an embodiment, delivery apparatus may be composed of a biocompatible material, such as a synthetic polymer(s) (water repelling materials like α-hydroxy acid [it is a family which consists of poly(lactic-co-glycolic) acid, PLGA], polyanhydrides, etc.), a naturally occurring polymer(s) (Polyvinylchloride (PVC), Polyethylene (PE), Polypropylene (PP), Polymethyl methacrylate (PMMA), Polystyrene (PS), Polytetrafluoroethylene (PTFE), Polyurethane (PU) and Polyamide (nylon), Polyethylene terephthalate (PET), Polyether sulfone (PES), etc.), composite material (plastic fiberglass, bone, etc.), ceramic material (lumina(Al2O3), Zirconia (ZrO2) (bioinert), Bioglass (Na2OCaOP2O3-SiO), etc.), and the like. In some embodiments, delivery apparatus 112 may be a hard biocompatible rod made of plastic or silicone. In some embodiments, the delivery apparatus may include an ECM.

Still referring to FIG. 1, in some embodiments, delivery apparatus 112 may include a subdermal rod. A “subdermal rod,” as used in this disclosure, is biocompatible material of such small dimensions that it can be fitted subcutaneously with at least an ordinary hypodermic needle. In some embodiments, the subdermal rod may be modeled after FDA-approved contraceptive devices, such subdermal implants Implanon and Nexplanon as produced by Merk, headquartered in Readington Township, NJ; Jadelle as produced by Bayer OY, headquartered in Finland; Vivelle Patch as produced by Novartis AG, headquartered in Basel, Switzerland; Alora patch as produced by Allergan, headquartered in Dublin, Ireland; Climara Patch as produced by Bayer, headquartered in Leverkusen, Germany; Delestrogen injection as produced by Par Sterile Products LLC, headquartered in Chestnut Ridge, N.Y.; Esclim patch as produced by Fournier Research, Inc., headquartered in New Jersey, United States; Estraderm patch as produced by Novartis, headquartered in Basel, Switzerland; Evamist skin spray as produced by KV Pharmaceutical, headquartered in St. Louis, MO; Femring as produced by Millicent Pharma, headquartered in East Hanover, NJ; Menostart patch as produced by Bayer; Minivelle patch as produced by Noven Pharmaceuticals, headquartered in Miami, Florida; and Vivelle-Dot patch as produced by Novartis. For example, the subdermal rod may be a plastic rod including an ethylene vinylacetate (EVA) copolymer core, containing 68 mg of the engineered ovarian cell, surrounded by an EVA copolymer skin. In some embodiments, the subdermal rod may be cylindrical or virtually cylindrical with a section of ranging between 1.5 and 2.0 mm, and a length between 1 and 5 cm. For example, the subdermal rod may be a 2″ long biocompatible plastic rod made of medical grade PVC. Additionally, the subdermal rod may contain an extracellular matrix encapsulating engineered ovarian support cell 104. For example, the extracellular matrix may be located in the core of the rod. In some embodiments, delivery apparatus 112 may include a membrane 116, micro needle patch, gel, cream, spray and the like. Active ingredients in the subdermal rod may include etonogestre, desogestrel, levonorgestrel, ethinylestradiol, mestranol, quinestrol, ethinylestradiol sulfonate, moxestrol, methylestradiol, estrogen esters, such as estradiol valerate, estradiol cypionate, estradiol acetate, estradiol benzoate, estradiol undecylate, and polyestradiol phosphate, progesterone, progestogen, acetomepregenol, algestone acetophenide, chlormadinone acetate, testosterone, and similar hormone medications. Inactive ingredients may include barium sulfate, magnesium stearate, ethylene-vinyl acetate copolymer, and other material described through this disclosure. Benefits of using a subdermal rod in hormone therapy: improved response when other treatments have failed and long-term bone protection, effectiveness in relieving menopausal symptoms in women who only respond to higher doses of HRT or in those who have had a surgical menopause, because the implants achieve higher levels of hormone in the circulation reversibility of long-term effects by removal of the rod. The ability of the rod to be easily removed plays a key role in preventing the long term effect correlated to systemic HRT wherein, medication is secreted and absorbed by the user at a fixed rate. Because the implant utilizes cyclic doing, a user is less likely to grow dependent and when the implant is removed, there may be little to no long-term effects contributed to irreversible or long-term HRT methods.

Still referring to FIG. 1, in some embodiments, implant 100 may include a membrane 116 containing a membrane surrounding delivery apparatus 112 for subdermal implantation of delivery apparatus 112. A “membrane,” as used in this disclosure is an outer layer of an object. Membrane 116 may be a selective barrier. For example, membrane 116 include epithelial membranes, serous membranes, connective tissue membranes, synovial membranes, and meninges. In some embodiments, membrane 116 may directly encapsulate an ECM containing engineered ovarian support cell 104 without delivery apparatus 112. To “encapsulate,” as used in this disclosure is to enclose a substance.

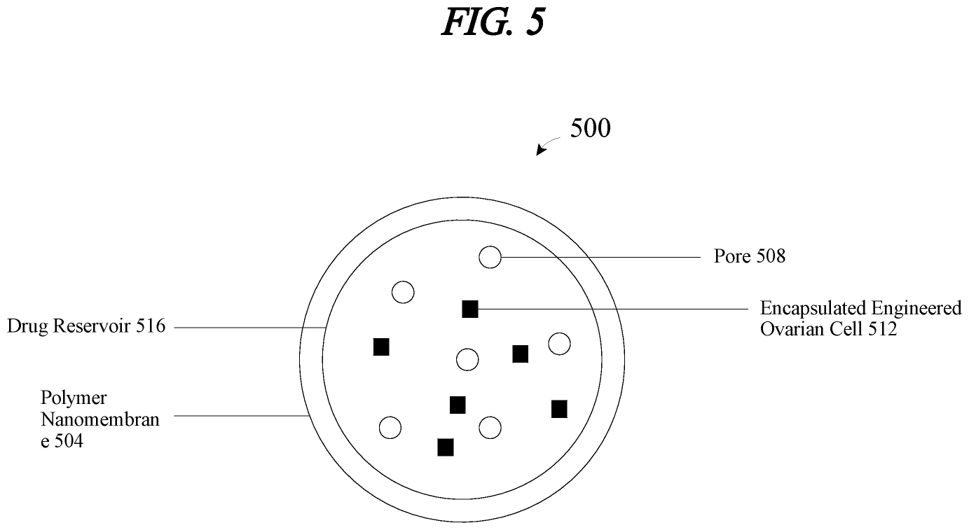

Encapsulation is commonly adopted in drug delivery systems to form a shell to protect a particular drug and prevent it from leaching out before reaching the targeted site. The outer layer may be made of hydrogel matrices or polymeric nanostructures that are able to entrap the targeted drug, as is described further below. Membrane 116 may include any extracellular matrix substrate, alone or in combination, as disclosed above. Additionally, extracellular matrix substrate may include any material used in membrane 116 as described throughout this disclosure. In some embodiments, membrane 116 may include polymers as described above. In some embodiments, membrane 116 may include a polymer matrix composite. “Polymer matrix composites (PMCs),” as used in this disclosure, are materials that uses organic polymer as matrix and fiber as reinforcement. There are different types of PMCs according to the polymer types like thermoset and thermoplastic polymers, epoxy, polymers other than epoxy, amorphous and semi crystalline polymers. In some embodiments, membrane 116 may include one or more perforated sheets. A “perforated sheet,” as used in this disclosure, is a nanomembrane with one or more openings acting as a passageway. For example, membrane 116 may be a polymer extracellular matrix, wherein the end of the patch is the side to come into direct contact with a user's hormonal environment. Perforated sheets may exhibit one or more qualities such as being freely suspended on the centimeter scale, exhibiting high mechanical strength, containing low surface energies and/or a sharp permeability cutoff at a hydrodynamic diameter of 10 nm suitable for protein separations. Perforated sheet may include any perforated sheet as described below in more detail in reference to FIG. 5.

Still referring to FIG. 1, the pore (perforation) size of membrane 116 may allow for bi-directional flow of small molecules, such as the secreted hormones of engineered ovarian support cell 104, but not migration of cells out of delivery apparatus 112. “Bi-directional flow,” as used in this disclosure, is the selective passage of a substance in both directions. A substance may be any substance as described throughout this disclosure, such as estrogen. For example, the pore size may be 25 microns in diameter while the cell is 81 microns. In some embodiments, membrane 116 may be a hard or closed biocomptible plastic or silicone device that keeps the cells encapsulated while allowing communication between engineered ovarian support cell 104 and the patient via the bloodstream. For example, the membrane 116 may act as an ectopic ovarian hub and sit on top of the user's skin. Once in contact with a user's hormonal environment, membrane 116 may allow bidirectional transmission of mechanical and biochemical signals across the patch membrane which may lead to a cooperative regulation of engineered ovarian support cell 104 functions, such as secretion of a hormone. Regarding implantation, when membrane 116 is in contact with the bloodstream, the patch may integrate into the bi-directional signaling of the user's brain and ovaries and only secretes the hormones signaled for production in lieu of the ovaries. In some embodiments, implantation of delivery apparatus 112 is reversible unlike in typical methods of hormone replacement therapy. “Reversible,” as used in this disclosure, is the removal of an implant from a user. Reversibility may include the short-term effect and/or half-life of the secreted hormones in a user. Implant 100 may be reversible in a minor procedure so that all cells will remain in the polymer and can be removed if needed (e.g., in the case of estrogen-dependent cancer), in contrast to injected agents which disperse through the patient's system. The fixed does of hormones administered in HRT do not replicate the physiological relationship between the brain and the ovaries and may lead to one or more effects due to prolonged use or imbalance of hormones administered. For example, HRT side effects may include breast tenderness, headaches, indigestion, abdominal (tummy) pain, vaginal bleeding, breast cancer (small but increased risk), deep vein thrombosis (small but increased risk), uterine lining growth, which can increase the risk of uterine cancer, Alzheimer's, stroke, heart attack, and similar conditions. Even with the most precise dose calculators, it would be nearly impossible to continuously measure blood markers and lifestyle factors and alter the dose in real-time using current methods of HRT. In addition, the pharmacokinetic properties such as the half-life of one or more hormones delivered via traditional HRT methodologies such as via a transdermal cream may allow hormones to continue to circulate in the body for days after cessation.

### Example 2: Method of Manufacturing an Implant for Ovarian Decline

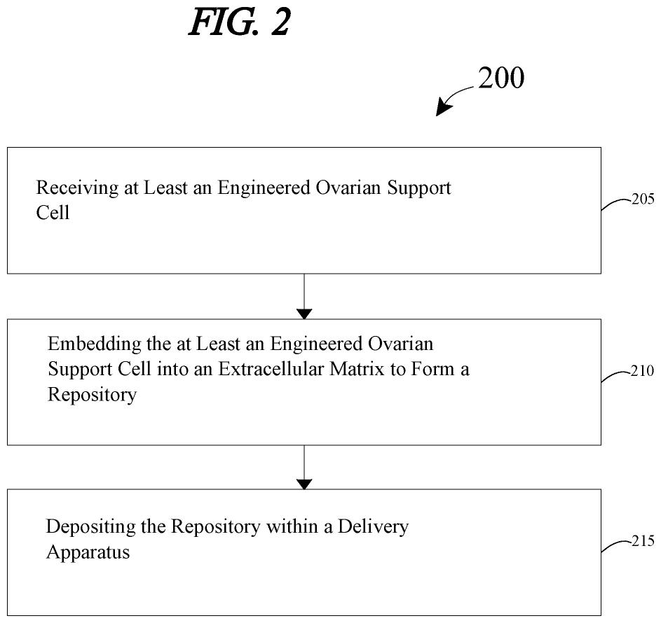

Referring now to FIG. 2, is an exemplary flow diagram of a method 200 for manufacturing an implant 100 for ovarian decline is illustrated. At step 205, method 200 includes receiving at least an engineered ovarian support cell. An engineered ovarian support may be any engineered ovarian support as defined and described in FIG. 1. For example, the engineered ovarian support cell may be a steroidogenic granulosa cell genetically modified and/or altered, using CRISPR technology, to express a certain quantity of hormones and/or a certain type of hormones. An engineered ovarian support cell may secrete levels as described herein (e.g., see Section B. Cyclic hormone release). Receiving the engineered ovarian support cell may including maturation of an oocyte received to a user in vitro. In some embodiments, receiving the engineered ovarian cell may include donation of an oocyte from a person not related to the user. Ovarian cells may be engineered in vitro using cellular cultivation methods. In some embodiments, engineering and ovarian support cell may involve ovarian cryopreservation has been done for decades, resulting in restoration of hormonal balance. Ovarian cryopreservation involves surgically extracting part of the ovary and freezing the ovarian cell-based tissue patches for reimplantation later in life. The reinsertion of the autologous ovarian patch leads to restoration of hormonal cycles and even spontaneous fertility. Many successful pregnancies have resulted from ovarian cryopreservation. This process is most commonly done to preserve future fertility and hormone production in prepubescent girls undergoing chemotherapy. Studies have found that, of women who have undergone ovarian cryopreservation, 62% conceive spontaneously, 64% have prolonged ovarian endocrine function, and 37% have a live birth.

At step 210, method 200 includes embedding the at least an engineered ovarian support cell into an extracellular matrix to form a repository. A “repository,” as used in this disclosure, is a dwelling used to store one or more cells. The repository may be perforated wherein the size of the pores may allow for bi-directional flow of small molecules, such as the secreted hormones of engineered ovarian support cell, but not migration of the engineered cells out of the repositor. In addition, the repository may allow for cyclic secretion of one or more hormones expressed and/or secreted by one or more engineered ovarian support cells. The extracellular matrix may be one as disclosed above, for example and with reference to FIG. 1. Embedding may include cell encapsulation methods such as emulsion, microfluidics, lithography, extrusion, and any other method one skilled in the art may understand as applicable. Encapsulation by emulsion may be performed by dispersing a hydrogel precursor into a non-miscible phase. Surfactants can be used to stabilize the emulsion and to obtain smaller drops (microemulsion). When the dispersion reaches equilibrium, the polymer drops are hardened according to the sol-gel mechanism of the hydrogel. Regarding microfluidics, it is a technique dealing with the handling of fluids in microenvironments, such as microchannels where the flow of fluids is generally laminar. The flow is characterized by low Reynolds numbers, meaning that it is dominated by viscous stresses with negligible inertia effects. The laminar flow allows a fine control over the characteristics of the microdrop. The generation of the microdrops may include the formation of emulsions of the polymer droplets in a non-miscible continuous phase. The generation of individual drops through microfluidics can be seen as a bottom-up approach to emulsification, compared with standard emulsification techniques being the top-down approach. In this bottom-up approach, a suspension of hydrogel precursor and cells is injected into a microchannel and the droplets are formed when the suspension intersects the continuous phase coming from other inlets. The intersection between the channels can have different geometries, with the T-junction and the flow-focusing being the most common.

Still referring to FIG. 2, cell encapsulation may include litography including but not limited to soft lithography and/or photolithography. Soft lithography is a strategy based on self-assembly and replica molding for carrying out micro- and nanofabrication. In this technique, a polymer solution may be poured or spin-coated onto a master and then is cross-linked, obtaining a rubbery replica. The replica may contain channels that can be filled with a suspension of a hydrogel precursor and cells. The channels are usually filled by pressing the replica onto the cell suspension. Regarding Extrusion, method to encapsulate cells into hydrogel beads includes gravitational dripping. A suspension of hydrogel precursor and cells is extruded through a small tube (i.e., a needle), the drop grows and when it reaches a critical mass it freely falls into a suitable hardening bath. This method usually leads to capsules above 1 mm diameter and because of the impact onto the bath they are not always spherical.

Still referring to FIG. 2, at step 215, method 200 includes depositing the repository within a delivery apparatus 112. A delivery apparatus may include any delivery apparatus as described above in more detail in FIG. 1. Deposition may occur by submerging delivery apparatus 112, such as a subdermal rod, into the repository and allowing the delivery apparatus to soak and fill up with the repository. In some embodiments, the repository may be injected into the delivery apparatus using a device, such as a syringe.

### Example 3: Method for Reversing Ovarian Decline

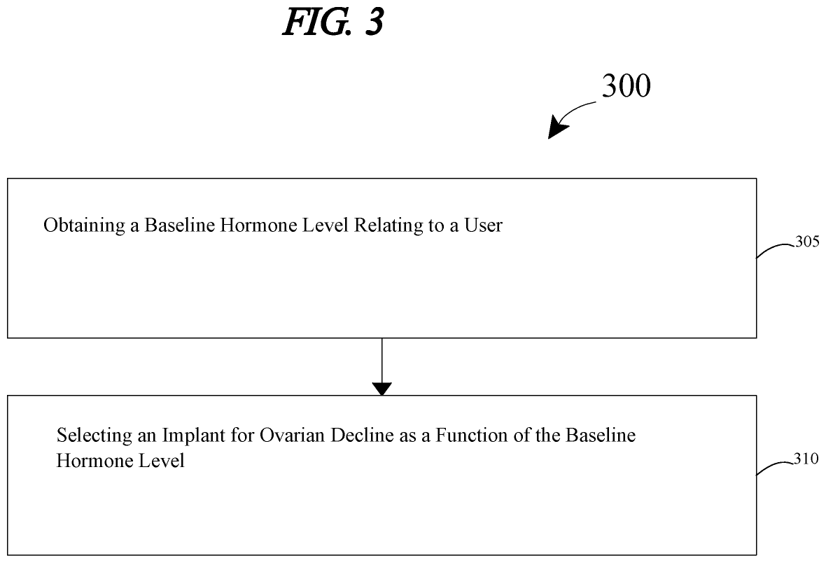

Referring now to FIG. 3, is an exemplary flow diagram of a method 300 for reversing ovarian decline is illustrated. At step 305, method 300 includes obtaining a baseline hormone level relating to a user. A “hormone level,” as used in this disclosure, is a quantitative value representing a level of one or more hormones of a user. The baseline hormone level may include quantitative values of estradiol (E2), luteinizing hormone (LH), follicle-stimulating hormone (FSH), progesterone (P4), estrone (E1), estriol (E3), testosterone, androgens, dehydroepiandrosterone (DHEA), triiodothyronine (T3), tetraiodothyronine (T4), calcitonin, melatonin, insulin, cortisol, human growth hormone (HGH), adrenaline levels and the like. In some embodiments, the baseline of hormone levels may be based on a blood analysis relating to a user. For example, blood analysis may include plasma hormone analysis techniques. In some embodiments, the baseline of hormone levels may be based on saliva hormone testing techniques. The baseline of hormone levels may be based on other forms of analysis such as hair, urine, and the like. At step 310, method 300 includes selecting an implant 100 for ovarian decline as a function of the baseline hormone level. In some embodiments, implant 100 may include at least an engineered ovarian support cell; an extracellular matrix substrate; and a delivery apparatus, for example and with reference to FIGS. 1 and 2. The implant selected may be an implant including the engineered ovarian cells configured to secrete hormones to replenish low levels of hormones relating to a user relating to the obtained baseline hormone levels. The baseline hormone levels may dictate the quantity and types of engineered ovarian support cells an implant may have to secrete hormone doses to adjust a user's hormone level as needed. The baseline hormone levels may be used to monitor the efficiency of ovarian decline reversal in a patient after implantation of the OSC implant.

### Example 4: Method for Administering an Implant for Ovarian Decline

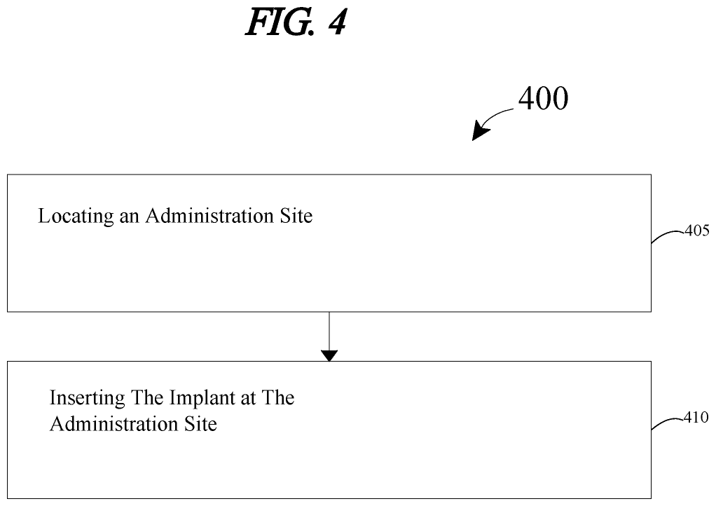

Referring now to FIG. 4, is an exemplary flow diagram of a method 400 for administering an implant for ovarian decline. The implant may be any implant as described throughout this disclosure. For example, the implant may be implant 100 as described in FIG. 1. At step 405, method 400 includes locating an administration site. As used in this disclosure, an “administration site,” is the location of application of the implant on a user. For example, the administration site for an IUD Implant may be the uterus. The administration site for a transdermal patch may include but is not limited to the upper chest, upper arm, lower abdomen, or hip of a user. In some embodiments, the implant may be a subdermal rod surrounded by a patch membrane as described in FIG. 1, wherein the administration locating the administration site includes identifying a location to accommodate bi-directional flow from the implant. This may be a site that is close to blood vessels.

For example, the administration site may be under the epidermis just above the dermis layer of a user's skin. The epidermis is the thin, outer layer of the skin that is visible to the eye and works to provide protection for the body. This part of the skin does not contain any blood vessels and is, therefore, dependent on the dermis. The epidermis consists of stratified squamous epithelial cells. There are four layers of the epidermis, according to the maturation of the cells: stratum germinativum, stratum spinosum, stratum granulosum, and stratum corneum. The “dermis,” as used in this disclosure is the layer of skin that lies beneath the epidermis and above the subcutaneous layer. It is the thickest layer of the skin and is made up of fibrous and elastic tissue. Thus, it provides strength and flexibility to the skin. The dermis includes two layers: the papillary dermis and the reticular dermis. The papillary dermis is the more superficial of the two and lies just beneath the epidermal junction. It is relatively thin and is made up of loose connective tissue, which includes capillaries, elastic fibers, reticular fibers, and collagen, The reticular dermis is the deeper and thicker layer of the dermis, which lies above the subcutaneous layer of the skin. It contains dense connective tissue, which includes blood vessels, elastic fibers (interlaced), collagen fibers (in parallel layers), fibroblasts, mast cells, nerve endings, and lymphatics. Additionally, there is a substance surrounding the components in the dermis. This has a gel-like consistency because it contains mucopolysaccharides, chondroitin sulfates, and glycoproteins. As there is no direct blood supply to the epidermis, this outermost layer of the skin relies on the underlying dermis, for the supply of nutrients and disposal of waste products. This occurs via a process of diffusion through the dermo-epidermal junction, which lies just below the stratum germinativum of the epidermis. An epidermis administration site may allow for bi-directional signaling and transmission of secreted hormones from the implant into the dermis layer containing the bloods vessels, a hormonal environment.

Still referring to FIG. 4, at step 410, method 400 includes inserting the implant at the administration site. In some embodiments, the implant may be inserted sub dermally in the upper arm. To reduce the risk of neural or vascular injury, the implant may be inserted at the inner side of the non-dominant upper arm about 8-10 cm (3-4 inches) above the medial epicondyle of the humerus. Insertion may include using an application such as, a hypodermic needle, vaginal cylinder applicator, ovoid's applicator, and similar devices. In some embodiments, the applicator may be modeled after the implant applicator used in applying Nexplanon and Implanon, respectively. Insertion may follow safety protocols applied by healthcare professionals. For example, confirming medical history of the user, sterilizing and numbing the administration site, and similar steps.

### Example 5: Extracellular Matrix (ECM) for Encapsulating the Engineered OSC

Referring now to FIG. 5, is an exemplary embodiment of an ECM for encapsulating the engineered ovarian support cell. ECM may be a polymer nanomembrane 504. Polymer nanomembranes 504 are synthetic structures with thickness less than 100 nm. Nanomembranes may be made from polymers such as Polyacrylonitrile (PAN), Polyethylene (PE), Polyvinylidene Fluoride (PVDF), polyamide, Polypropylene, and polyether sulfone (PES). Their surface-area-volume ratio increases to at least a few orders of magnitude. These membranes have pore size in the range of 1nm to 300 nm. Nanomembranes allow for regulation of the physio-chemical properties of membrane pores 508, which make them attractive for controlling drug delivery rates. Membranes are used to control the rate of delivery of drugs to the body as well as drug permeation from the reservoir to attain the required rate of drug delivery.

Therefore, drug delivery is controlled by both passive diffusion and biodegradation mechanisms.

Membranes can carry one or more bioactive agents and have been developed into different classes of carriers. These different carriers can be carbon-based nanomaterials, polymeric membranes, and inorganic membranes, where the bulk properties of the membrane are governed by its building blocks, i.e., the nanoparticles. As used in this disclosure, a “drug encapsulated engineered ovarian cell” is an engineered ovarian support cell encapsulated by a membrane and/matrix. Here, a drug encapsulated engineered ovarian cell 512 may be held in the compartment of a drug reservoir 516, in which the surface of the drug-releasing layer is covered by a rate-controlled polymeric membrane. A “drug reservoir,” as used in the disclosure, is the homogenous dispersion of drug within a matrix. A drug reservoir may include a concentration of one or more ingredients including but not limited to an ovarian support cell, a prescription drug product, a supplement, a chemical ingredient and the like. For example, a drug may include a plurality of encapsulated engineered ovarian cells 516. Drug reservoir 516 may be a solid, solid dispersion, or a drug solution in liquid form. Pore 508 size of the membrane may allow for transmission of hormones secreted from encapsulated engineered ovarian support cell 512 out of the ECM and into the hormonal environment of the user in a bi-directional flow as described above in more detail. In an embodiment, pore 508 may not allow for an engineered ovarian support cell 512 to pass, thereby preventing bi-directional flow of the engineered ovarian support cell.

The encapsulation process for preparing the drug formulation (e.g., an OSC implant of the disclosure) inside the reservoir compartment includes fabrication by microencapsulation, coating, and molding techniques. Microencapsulation is a process in which tiny particles or droplets are surrounded by a coating to give small capsules, with useful properties. In general, it is used to incorporate food ingredients, enzymes, cells or other materials on a micro metric scale. Microencapsulation can also be used to enclose solids, liquids, or gases inside a micrometric wall made of hard or soft soluble film, to reduce dosing frequency and prevent the degradation of pharmaceuticals. In its simplest form, a microcapsule is a small sphere comprising a near-uniform wall enclosing some material. The enclosed material in the microcapsule is referred to as the core, internal phase, or fill, whereas the wall is sometimes called a shell, coating, or membrane. Some materials such as but not limited to lipids and polymers, such as alginate, may be used as a mixture to trap the material of interest inside. Most microcapsules have pores with diameters between a few nanometers and a few micrometers. Materials generally used for coating are Ethyl, cellulose, Polyvinyl, alcohol, Gelatin, Sodium alginate. Microencapsulation can be used to slow the release of a drug (e.g., a hormone) into the body. This may permit one controlled release dose to substitute for several doses of non-encapsulated drug and may decrease toxic side effects for some drugs by preventing high initial concentrations in the blood. There is usually a certain desired release pattern. In some cases, it is zero-order, i.e., the release rate is constant. In this case, the microcapsules deliver a fixed amount of drug per minute or hour during the period of their effectiveness. This can occur as long as a solid reservoir or dissolving drug is maintained in the microcapsule. Additionally, a thin layer of silicone or poly acrylate adhesive may be applied to the external surface of the rate controlling membrane to achieve intimate contact of the ECM and the skin surface. The polymeric membrane may be manufactured from a nonporous, microporous semipermeable membrane.

### Example 6: Cyclic Dosing Schedule

Referring now to FIG. 6, is an exemplary diagram of a cyclic dosing schedule. The implant 100, as disclosed at least in FIG. 1, may work in tandem with the ovaries to produce sufficient hormone levels. For example, a user in ovarian decline may still produce estrogen but not at sufficient levels, the implant 100 may secrete a dose of estrogen that matches the difference. In some embodiments, the implant may be the sole source of the hormone, in lieu of the ovary. On average, levels of estradiol in premenopausal women are highly variable throughout the menstrual cycle and reference ranges widely vary from source to source. Estradiol levels are minimal and according to most laboratories range from 20 to 80 pg/ml during the early to mid-follicular phase (or the first week of the menstrual cycle, also known as menses). Levels of estradiol gradually increase during this time and through the mid to late follicular phase (or the second week of the menstrual cycle) until the pre-ovulatory phase. At the time of pre-ovulation (a period of about 24 to 48 hours), estradiol levels briefly surge and reach their highest concentrations of any other time during the menstrual cycle. Circulating levels are typically between 130 and 200 pg/ml at this time, but in some women may be as high as 300 to 400 pg/ml, and the upper limit of the reference range of some laboratories are even greater (for instance, 750 pg/ml). Following ovulation (or mid-cycle) and during the latter half of the menstrual cycle or the luteal phase, estradiol levels plateau and fluctuate between around 100 and 150 pg/ml during the early and mid-luteal phase, and at the time of the late luteal phase, or a few days before menstruation, reach a low of around 40 pg/ml.

Still referring to FIG. 6, in women, progesterone levels are relatively low during the preovulatory phase of the menstrual cycle, rise after ovulation, and are elevated during the luteal phase, as shown in the diagram above. Progesterone levels tend to be less than 2 ng/ml prior to ovulation and greater than 5 ng/ml after ovulation. If pregnancy occurs, human chorionic gonadotropin is released, maintaining the corpus luteum and allowing it to maintain levels of progesterone. Between 7 and 9 weeks, the placenta begins to produce progesterone in place of the corpus luteum in a process called the luteal-placental shift. Luteinizing hormone (LH) levels are normally low during childhood and in women, high after menopause. Since LH is secreted as pulses, it is necessary to follow its concentration over a sufficient period of time to get proper information about its blood level. During reproductive years, typical levels are between 1 and 20 IU/L. Physiologic high LH levels are seen during the LH surge and typically last 48 hours. Follicle stimulating hormone levels are typically measured in the early follicular phase of the menstrual cycle, day three to five, counted from last menstruation. At this time, the levels of estradiol (E2) and progesterone are at the lowest point of the menstrual cycle. FSH levels in this time is often called basal FSH levels, to distinguish from the increased levels when approaching ovulation. The mean values for women before ovulation are around (3,8-8,8) mUI/ml. After ovulation these levels drop to between (1,8-5,1) mUI/ml. At the mid of the menstrual cycle, it reaches its highest value, between (4.5-22.5) mlU / mL. During menopause, the values go up even more, between (16.74-113.59)

Still referring to FIG. 6, regarding the cyclic dosing schedule, during the first week of user's menstrual cycle, and/or the follicular phase, FSH may primarily be secreted by the engineered ovarian cell through the implant and into a user's blood stream. The follicular phase starts on the first day of menstruation and ends with ovulation. Prompted by the hypothalamus, the pituitary gland releases follicle stimulating hormone (FSH). This hormone stimulates the ovary to produce around 5 to 20 follicles (tiny nodules or cysts), which bead on the surface. During this phase, 551-2753 pmol/L of E2 may also be secreted from the implant. During the second week and/or ovulation phase of the menstrual cycle, LH may primarily be secreted by the engineered ovarian cell through the implant and into a user's blood stream. Ovulation is the release of a mature egg from the surface of the ovary. This usually occurs mid-cycle, around two weeks or so before menstruation starts. During the follicular phase, the developing follicle causes a rise in the level of estrogen. The hypothalamus in the brain recognizes these rising levels and releases a chemical called gonadotrophin-releasing hormone (GnRH). This hormone prompts the pituitary gland to produce raised levels of luteinizing hormone (LH) and FSH. During ovulation, the egg bursts from its follicle, but the ruptured follicle stays on the surface of the ovary. For the next two weeks or so, the follicle transforms into a structure known as the corpus luteum. This structure starts releasing progesterone, along with small amounts of estrogen. This combination of hormones maintains the thickened lining of the uterus, waiting for a fertilized egg to stick (implant). In this phase of ovulation, implant 100 may primarily secrete 2 to 25 ng/ml of progesterone. Secretion of a hormone may stop based on chemical singling of the hypothalamus through the blood stream.

### Example 7: Production Rates of Estradiol (E2) From Engineered OSCs

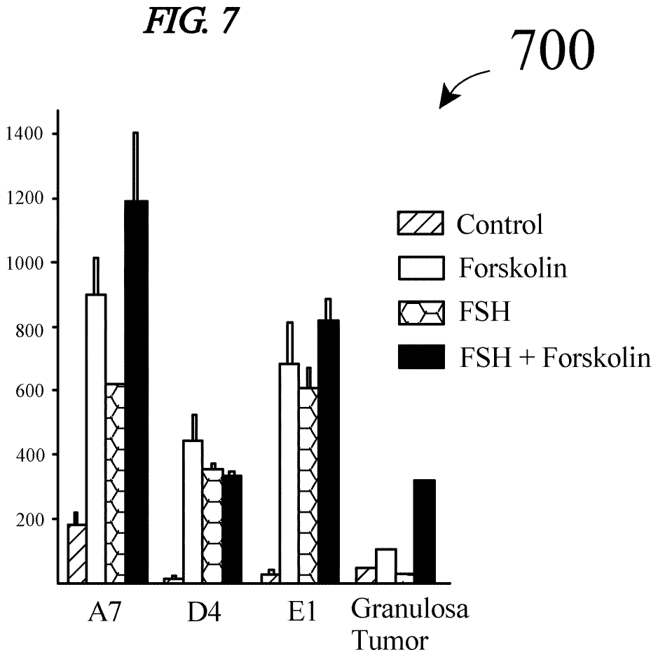

Referring now to FIG. 7, an exemplary graph 700 illustrating production rates of estradiol (E2) from engineered ovarian support cells configured to produce one or more hormones prevalent in ovarian health is illustrated. E2 levels were measured in pg/uL. Graph shows the E2 production rate from three engineered ovarian support cells: A7, D4, E1 compared to a granulosa tumor in response to stimuli.

Stimuli includes control, forskolin, FSH, and FSH+Forskolin. A7 contained NR5A1 and RUNX1. D4 contained NR5A1, RUNX1, GATA4, and FOXL2. E1 contained NR5A1, RUNX1, and GATA4. The control contained basal media. The granulosa tumor is a tumor that arises from granulosa cells. It is estrogen secreting and present as large, complex, ovarian mass. The granulosa tumor is part of the sex cord-gonadal stromal tumor or non-epithelial group of tumors. Regarding the forskolin stimulus, forskolin (coleonol) is a labdane diterpene produced by the plant Coleus barbatus (Blue Spur Flower). Forskolin contains some unique functional elements, including the presence of a tetrahydropyran-derived heterocyclic ring. Forskolin is commonly used in biochemistry to raise levels of cyclic AMP (cAMP) in the study and research of cell physiology. Forskolin activates the enzyme adenylyl cyclase and increases intracellular levels of cAMP. cAMP is an important second messenger necessary for the proper biological response of cells to hormones and other extracellular signals. It is required for cell communication in the hypothalamus/pituitary gland axis and for the feedback control of hormones via induction of corticotropin-releasing factor gene transcription. Cyclic AMP acts by activating cAMP-sensitive pathways such as protein kinase A and EPAC1. Its derivatives include colforsin daropate, NKH477, and FSK88, which may be more potent than forskolin at raising cAMP. These derivatives may have pharmaceutical utility against bronchoconstriction and heart failure. Regarding the FSH stimulus, Follicle-stimulating hormone (FSH) is a gonadotropin, a glycoprotein polypeptide hormone. FSH is synthesized and secreted by the gonadotropic cells of the anterior pituitary gland and regulates the development, growth, pubertal maturation, and reproductive processes of the body. FSH stimulates the growth and recruitment of immature ovarian follicles in the ovary. FSH initiates follicular growth, specifically affecting granulosa cells.

Still referring to FIG. 7, the engineered cells exposed to the control produced up to 200 pg/uL of E2. When exposed to Forskolin, around 900 pg/uL of E2. In response to FSH, up to pg/uL of E 2. In response to FSH+Forskolin, up to 1200 pg/uL of E2. A7 showed the most significant E2 production rates, while the granulosa tumor, at the most, produced around 300 pg/uL of E2 when exposed to FSH+Forskolin.

The foregoing has been a detailed description of illustrative embodiments of the invention.

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, apparatuses, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

### SPECIFIC EMBODIMENTS

Several non-limiting, exemplary embodiments of the disclosure are enumerated below. The below embodiments should not be construed to limit the scope of the invention, rather, the below are presented as some examples of the utility of the invention.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

1. An implant comprising (i) a population of ovarian support cells (OSCs) and (ii) an extracellular matrix substrate.

2. The implant of claim 1, wherein the population of OSCs comprises one or more ovarian granulosa cells, ovarian lutein cells, and/or ovarian theca cells.

3. The implant of claim 2, wherein the population of OSCs comprises one or more ovarian granulosa cells.

4. The implant of claim 3, wherein the one or more ovarian granulosa cells express one or more, or all, of proteins FOXL2, CD82, follicle-stimulating hormone receptor (FSHR), FOXL2, NR5A1, GATA4, RUNX1, and RUNX2.

5. The implant of claim 3 or 4, wherein the implant comprises from about 1×10⁶ and to about 1×10⁷ ovarian granulosa cells.

6. The implant of claim 5, wherein the implant comprises from about 1×10⁶ to about 2×10⁶ ovarian granulosa cells, from about 2×10⁶ to about 3×10⁶ ovarian granulosa cells, from about 3×10⁶ to about 4×10⁶ ovarian granulosa cells, from about 4×10⁶ to about 5×10⁶ ovarian granulosa cells, from about 5×10⁶ to about 6×10⁶ ovarian granulosa cells, from about 6×10⁶ to about 7×10⁶ ovarian granulosa cells, from about 7×10⁶ to about 8×10⁶ ovarian granulosa cells, from about 8×10⁶ to about 9×10⁶ ovarian granulosa cells, or from about 9×10⁶ to about 1×10⁷ ovarian granulosa cells.

7. The implant of claim 5, wherein the implant comprises about 1×10⁶ ovarian granulosa cells, about 2×10⁶ ovarian granulosa cells, about 3×10⁶ ovarian granulosa cells, about 4×10⁶ ovarian granulosa cells, about 5×10⁶ ovarian granulosa cells, about 6×10⁶ ovarian granulosa cells, about 7×10⁶ ovarian granulosa cells, about 8×10⁶ ovarian granulosa cells, about 9×10⁶ ovarian granulosa cells, or about 1×10⁷ ovarian granulosa cells.

8. The implant of any one of claims 3-7, wherein the one or more ovarian granulosa cells secrete an estrogen.

9. The implant of claim 8, wherein the estrogen is estradiol.

10. The implant of any one of claims 2-9, wherein the population of OSCs comprises one or more ovarian lutein cells.

11. The implant of claim 10, wherein the one or more ovarian lutein cells express one or more, or all, of proteins KRT19, CYP19A1, STAR, CYP17A1, and PGR.

12. The implant of claim 10 or 11, wherein the implant comprises from about 1×10⁶ and to about 1×10⁷ ovarian lutein cells.

13. The implant of claim 12, wherein the implant comprises from about 1×10⁶ to about 2×10⁶ ovarian lutein cells, from about 2×10⁶ to about 3×10⁶ ovarian lutein cells, from about 3×10⁶ to about 4×10⁶ ovarian lutein cells, from about 4×10⁶ to about 5×10⁶ ovarian lutein cells, from about 5×10⁶ to about 6×10⁶ ovarian lutein cells, from about 6×10⁶ to about 7×10⁶ ovarian lutein cells, from about 7×10⁶ to about 8×10⁶ ovarian lutein cells, from about 8×10⁶ to about 9×10⁶ ovarian lutein cells, or from about 9×10⁶ to about 1×10⁷ ovarian lutein cells.

14. The implant of claim 12, wherein the implant comprises about 1×10⁶ ovarian lutein cells, about 2×10⁶ ovarian lutein cells, about 3×10⁶ ovarian lutein cells, about 4×10⁶ ovarian lutein cells, about 5×10⁶ ovarian lutein cells, about 6×10⁶ ovarian lutein cells, about 7×10⁶ ovarian lutein cells, about 8×10⁶ ovarian lutein cells, about 9×10⁶ ovarian lutein cells, or about 1×10⁷ ovarian lutein cells.

15. The implant of any one of claims 10-14, wherein the one or more ovarian lutein cells secrete a progestogen.

16. The implant of claim 15, wherein the progestogen is progesterone.

17. The implant of any one of claims 2-16, wherein the population of OSCs comprises one or more ovarian theca cells.

18. The implant of claim 17, wherein the one or more ovarian theca cells express one or both of proteins NR2F2 and GATA4.

19. The implant of claim 17 or 18, wherein the implant comprises from about 1×10⁶ and to about 1×10⁷ ovarian theca cells.

20. The implant of claim 19, wherein the implant comprises from about 1×10⁶ to about 2×10⁶ ovarian theca cells, from about 2×10⁶ to about 3×10⁶ ovarian theca cells, from about 3×10⁶ to about 4×10⁶ ovarian theca cells, from about 4×10⁶ to about 5×10⁶ ovarian theca cells, from about 5×10⁶ to about 6×10⁶ ovarian theca cells, from about 6×10⁶ to about 7×10⁶ ovarian theca cells, from about 7×10⁶ to about 8×10⁶ ovarian theca cells, from about 8×10⁶ to about 9×10⁶ ovarian theca cells, or from about 9×10⁶ to about 1×10⁷ ovarian theca cells.

21. The implant of claim 19, wherein the implant comprises about 1×10⁶ ovarian theca cells, about 2×10⁶ ovarian theca cells, about 3×10⁶ ovarian theca cells, about 4×10⁶ ovarian theca cells, about 5×10⁶ ovarian theca cells, about 6×10⁶ ovarian theca cells, about 7×10⁶ ovarian theca cells, about 8×10⁶ ovarian theca cells, about 9×10⁶ ovarian theca cells, or about 1×10⁷ ovarian theca cells.

22. The implant of any one of claims 17-21, wherein the one or more ovarian theca cells secrete an androgen.

23. The implant of claim 22, wherein the androgen is androstenedione.

24. The implant of any one of claims 2-23, wherein the OSCs comprise one or more ovarian granulosa cells, ovarian lutein cells, and ovarian theca cells, and wherein the ratio of ovarian granulosa cells to ovarian lutein cells to ovarian theca cells in the implant is from about 10:1:1 to about 1,000:1:1.

25. The implant of claim 24, wherein the ratio of ovarian granulosa cells to ovarian lutein cells to ovarian theca cells in the implant is about 100:1:1.

26. The implant of any one of claims 1-25, wherein the one or more OSCs are obtained by differentiating a population of induced pluripotent stem cells (iPSCs) into one or more ovarian granulosa cells, ovarian lutein cells, and/or ovarian theca cells.

27. The implant of any one of claims 1-26, wherein the extracellular matrix substrate comprises one or more of collagen, an epidermal growth factor (EGF), an elastin, a fibronectin, a vitronectin, a laminin, a cell adhesion protein, or a plant-derived protein or protein polymer.

28. The implant of claim 27, wherein the collagen is fibrilar collagen.

29. The implant of claim 27 or 28, wherein the collagen is collagen type I, type II, type III, type V, type XI.

30. The implant of any one of claims 27-29, wherein the elastin is tropoelastin or mature elastin.

31. The implant of any one of claims 27-30, wherein the plant-derived protein polymer is alginate.

32. A method of increasing secretion of an estrogen, progestogen, and/or androgen in a human subject, the method comprising administering to the subject the implant of any one of claims 1-31.

33. The method of claim 32, wherein the subject is a pre-menopausal human subject, optionally wherein administration of the implant restores secretion of an estrogen, progestogen, and/or androgen in the subject.

34. The method of claim 32 or 33, wherein the subject is diagnosed as having ovarian dysfunction.

35. The method of any one of claims 32-34, wherein, prior to administration of the implant to the subject, the subject exhibits consistent, circulating estradiol levels of about 20 pg/ml or less, optionally wherein the subject exhibits consistent, circulating estradiol levels of from about 10 to about 20 pg/ml.

36. The method of any one of claims 32-35, wherein. prior to administration of the implant to the subject, the subject exhibits consistent, circulating progesterone levels of about 0.5 ng/ml or less, optionally wherein the subject exhibits consistent, circulating progesterone levels of from about 0.01 ng/ml to about 0.5 ng/ml.

37. The method of any one of claims 32-36, wherein the subject has previously undergone an ovariectomy.

38. The method of any one of claims 32-37, wherein prior to administration of the implant to the subject, the subject exhibits ovarian decline.

39. The method of claim 38, wherein the ovarian decline is a result of menopause.

40. The method of claim 38, wherein the ovarian decline is not a result of menopause, optionally wherein the ovarian decline is a result of premature menopause.

41. The method of any one of claims 38-40, wherein the ovarian decline is a result of a disease or condition selected from primary ovarian insufficiency (POI), polycystic ovarian syndrome (PCOS), endometriosis, uterine fibroids, gynecological cancer, interstitial cystitis, pelvic inflammatory disease (PID), vaginitis, cervical dysplasia, and pelvic floor prolapse.

42. The method of any one of claims 32-41, wherein the population of OSCs secretes estradiol during an early- to mid-follicular phase of the subject's menstrual cycle at a serum concentration of from about 20 pg/ml to about 80 pg/ml.

43. The method of claim 42, wherein the population of OSCs secretes estradiol during an early- to mid-follicular phase of the subject's menstrual cycle at a serum concentration of from about 20 pg/ml to about to 30 pg/ml, from about 30 pg/ml to about 40 pg/ml, from about 40 pg/ml to about 50 pg/ml, from about 50 pg/ml to about 60 pg/ml, from about 60 pg/ml to about 70 pg/ml, or from about 70 pg/ml to about 80 pg/ml.

44. The method of any one of claims 32-43, wherein the population of OSCs secretes estradiol during a mid- to late-follicular phase of the subject's menstrual cycle at a serum concentration of from about 20 pg/ml to about 200 pg/ml.

45. The method of claim 44, wherein the population of OSCs secretes estradiol during a mid- to late-follicular phase of the subject's menstrual cycle at a serum concentration of from about 20 pg/ml to about to 50 pg/ml, from about 50 pg/ml to about 70 pg/ml, from about 70 pg/ml to about 90 pg/ml, from about 90 pg/ml to about 110 pg/ml, from about 110 pg/ml to about 130 pg/ml, from about 130 pg/ml to about 150 pg/ml, from about 150 pg/ml to about t 170 pg/ml, or from about 170 pg/ml to about 200 pg/ml.

46. The method of any one of claims 32-45, wherein the population of OSCs secretes estradiol during a pre-ovulation phase of the subject's menstrual cycle at a serum concentration of from about 100 pg/ml to about 400 pg/ml.

47. The method of claim 46, wherein the population of OSCs secretes estradiol during a pre-ovulation phase of the subject's menstrual cycle at a serum concentration of from about 100 pg/ml to about to 150 pg/ml, from about 150 pg/ml to about 200 pg/ml, from about 200 pg/ml to about 250 pg/ml, from about 250 pg/ml to about 300 pg/ml, from about 300 pg/ml to about 350 pg/ml, or from about 350 pg/ml to about 400 pg/ml.

48. The method of any one of claims 32-47, wherein the population of OSCs secretes estradiol during an ovulation phase of the subject's menstrual cycle at a serum concentration of from about 250 pg/ml to about 500 pg/ml.

49. The method of claim 48, wherein the population of OSCs secretes estradiol during an ovulation phase of the subject's menstrual cycle at a serum concentration of from about 250 pg/ml to about to 300 pg/ml, from about 300 pg/ml to about 350 pg/ml, from about 350 pg/ml to about 400 pg/ml, from about 400 pg/ml to about 450 pg/ml, or from about 450 pg/ml to about 500 pg/ml.

50. The method of any one of claims 32-49, wherein the implant is administered to the subject in the form of a patch.

51. The method of any one of claims 32-50, wherein the implant is administered to the subject subdermally, optionally wherein the implant is inserted beneath a segment of skin on the subject's arm.

52. The method of any one of claims 32-50, wherein the implant is administered onto one, or both, of the subject's ovaries.

53. The method of any one of claims 32-50, wherein the implant is administered into the subject's uterus, optionally wherein the implant is embedded into the subject's uterine lining.

54. The method of any one of claims 1-53, wherein the implant is administered to the subject at a frequency of no greater than once every 6, 7, 8, 9, 10, 11, or 12 months.

55. The method of any one of claims 1-53, wherein the implant is administered to the subject at a frequency of no greater than once every 24, 36, 48, or 52 weeks.

56. The method of any one of claims 1-53, wherein the implant is administered to the subject at a frequency of no greater than once every one, two, or three years.

57. A kit comprising the implant of any one of claims 1-31 and a package insert.

58. The kit of claim 57, wherein the package insert instructs a user of the kit to perform the method of any one of claims 32-56.

59. An implant for ovarian decline, the implant comprising:
at least an engineered ovarian support cell;
an extracellular matrix substrate; and
a delivery apparatus.

60. The implant of claim 59, wherein the at least an engineered ovarian support cell further comprises an engineered granulosa cell.

61. The implant of claim 59, wherein the at least an engineered ovarian support cell further comprises an engineered lutein cell.

62. The implant of claim 59, wherein the at least an engineered ovarian support cell further comprises an engineered theca cell.

63. The implant of claim 59, wherein the at least an engineered ovarian support cell is engineered to secrete a hormone in a cyclic dose.

64. The implant of claim 59, wherein the extracellular matrix substrate further comprises a collagen polymer matrix.

65. The implant of claim 64, wherein the collagen polymer matrix is about 100-500 micron particles.

66. The implant of claim 64, wherein the collagen polymer matrix is about 500-800 micron cubes.

67. The implant of claim 64, wherein the collagen polymer matrix further comprises a perforated sheet.

68. The implant of claim 59, wherein the at least an engineered ovarian support cell is embedded within the extracellular matrix substrate.

69. The implant of claim 59, wherein the delivery apparatus further comprises a subdermal rod.

70. The implant of claim 59, further comprising a membrane surrounding the delivery apparatus.

71. The implant of claim 70, wherein the membrane is comprised within a patch.

72. The implant of claim 71, wherein the membrane allows diffusion of one or more macromolecules therethrough, optionally wherein the one or more macromolecules comprises one or more of an estrogen (e.g., estradiol), a progestogen (e.g., progesterone), and an androgen (e.g., androstenedione).

73. The implant of claim 59, wherein the membrane encapsulates the at least an engineered ovarian support cell.

74. The implant of claim 59, wherein the membrane provides bi-directional flow of a hormone.

75. The implant of claim 59, wherein the delivery apparatus is removable.

76. A method for manufacturing an implant for ovarian decline, the method comprising:
embedding at least an engineered ovarian support cell into an extracellular matrix to form a repository; and
depositing the repository within a delivery apparatus.

77. A method for reversing ovarian decline, the method comprising:
obtaining a baseline hormone level relating to a user; and
selecting an implant for ovarian decline as a function of the baseline hormone level.

78. The method of claim 77, wherein the implant further comprises:
at least an engineered ovarian support cell;
an extracellular matrix substrate; and
a delivery apparatus.

79. The method of claim 77, wherein selecting the implant further comprises selecting at least an engineered ovarian support cell as a function of the baseline hormone level.

80. The method of claim 79, wherein selecting the at least an engineered ovarian support cell further comprises selecting a cell quantity.
